(19) United States
(12) Patent Application Publication
(10) Pub. No.: US 2026/0232412 A1
(43) Pub. Date: Aug. 13, 2026

(54) SYSTEM AND METHOD FOR INTRAORAL SCAN REGISTRATION

(71) Applicant: 3SHAPE A/S, Copenhagen K (DK)

(72) Inventors: Christopher Prinds Bilberg, Copenhagen K (DK); Anders Gaarde, Copenhagen K (DK)

(73) Assignee: 3SHAPE A/S, Copenhagen K (DK)

(21) Appl. No.: 19/155,890

(22) PCT Filed: Feb. 12, 2024
(86) PCT No.: PCT/EP2024/053465

§ 371 Date: Aug. 12, 2025

(30) Foreign Application Priority Data
Feb. 13, 2023 (DK) PA 2023 70080

(51) Int. Cl. A61C 9/00; G06T 7/00; G06T 7/30; G06T 7/55; G06T 15/08; G06T 15/20; H04L 5/14

(52) U.S. Cl. CPC A61C 9/006; G06T 7/0012; G06T 7/30; G06T 7/55; G06T 15/08; G06T 15/20; H04L 5/14; G06T 2207/30036; G06T 2207/30168; G06T 2210/08; G06T 2210/41

(57) ABSTRACT

An intraoral scanning system including a handheld intraoral scanning device and one or more client devices is provided. The handheld intraoral scanning device captures a plurality of two-dimensional scan images during a scanning session of a dental arch. The handheld intraoral scanning device provides three-dimensional surface information based on the plurality of 2D scan images. The handheld intraoral scanning device establishes a connection to one or more wireless full-duplex communication channels. The one or more client devices establishes a connection to one of the one or more wireless full-duplex communication channels by forwarding an identification number to the handheld intraoral scanning device. The one or more client devices receives the 3D surface information and render the 3D surface information into an interactive 3D graphical representation.

### TECHNICAL FIELD

An example embodiment of the present invention generally relates to intraoral scan registration and more particularly relates to an intraoral scanning system and a method for intraoral scan registration based on edge computing.

### BACKGROUND OF THE INVENTION

Typically, intraoral scanners may be utilized by users, such as dentists in dental industry. For example, intraoral scans of dental arches of patients captured by the intraoral scanners may be utilized to create digital impressions of oral cavities of the patients. The intraoral scanners comprise a light source that is projected on the dental arches, to capture the intraoral scans. The intraoral scans are then processed to generate three-dimensional (3D) information (e.g., a 3D model) of the dental arches and the 3D model may be displayed on a screen for the dentist for examination.

Notably, a real-time feedback of the captured intraoral scans is essential for post-processing of the 3D model. Conventionally, the real-time feedback is collected by using a live reconstruction of the 3D model based on incoming sub-scans (such as the intraoral scans) from the intraoral scanner on a computing device having a significant processing capacity (for example, a power PC). In case registration of the 3D information (such as a point cloud) is lost, the dentist may be informed through a user interface (UI) (for example, the UI of the computing device). The dentist may then need to capture the intraoral scans again before finishing a scanning session of the dental arches to compensate for the lost 3D information.

A conventional pipeline of generating the 3D model using the intraoral scans may include capturing the sub-scans data and transmitting the captured sub-scans data to the computing device using a dedicated network. The transmitted sub-scans data may be separated into a texture and amplitude image on the computing device. Further, scanner calibration parameters and filtering are applied on the texture and amplitude image applied to the generate the point cloud data on the computing device. The registration of the point cloud between the sub-scans is further performed on the computing device to find a spatial transformation between each sub-scan and stitch individual point clouds together to reconstruct an accumulated 3D model. The 3D model may be rendered on the computing device.

The conventional method of generating the 3D model possess several disadvantages. For example, the process requires usage of external infrastructure to transmit the sub-scans data over the dedicated network. Moreover, there is a dependence on the computing device for several initial scans' registration generation of the 3D information. Given that a large amount of the sub-scans data needs to be sent over the dedicated network and be shown to the dentists in real-time, may put certain requirements on infrastructure of the dedicated network in dental clinics with respect to throughput and latency. Moreover, in case the infrastructure of the dedicated network is unable to fulfil the infrastructure requirements, the sub-scans data may get lost on the way or lag, thereby, making interaction of the dentist with the intraoral scanner difficult. Furthermore, there may be a limitation on a number of the intraoral scanners that may be connected with the dedicated network at a time in the dental clinics. Such limitations may become critical if part of the 3D model processing needs to be done on a cloud server. Generally, treatment rooms in the dental clinics may be unable to fulfill the hardware requirements needed for the intraoral scanning. Thus, the dental clinics may require additional computing devices or multiple treatment rooms may need to rely on the common computing devices. Moreover, the conventional method allows a single computing device to handle a single intraoral scanner at a time, thereby making parallel processing difficult. Therefore, there is a need of improved systems and methods of intraoral scanning to overcome the disadvantages of the conventional method.

### SUMMARY OF THE INVENTION

An intraoral scanning system, a method and a computer programmable product are provided for intraoral scan registration using a handheld intraoral scanning device and render of three-dimensional (3D) surface information on one or more client devices.

In one aspect, an intraoral scanning system is disclosed. The intraoral scanning system includes a handheld intraoral scanning device and one or more client devices. The handheld intraoral scanning device may be configured to capture a plurality of two-dimensional (2D) scan images during a scanning session of a dental arch. The handheld intraoral scanning device may further provide the 3D surface information based on the plurality of 2D scan images captured during the scanning session.

The 3D surface information may be provided in real time, such that a displaying unit is configured to display the 3D surface information in real time, and/or, such that the 3D surface information is transmitted wirelessly in real time.

The handheld intraoral scanning device may further include a web server interface configured to communicate via a web network and establish a connection to one or more wireless full-duplex communication channels. Furthermore, each of the one or more client devices is configured to establish a connection to one of the one or more wireless full-duplex communication channels by forwarding an identification number to the handheld intraoral scanning device via the web network. Each of the one or more client devices is further configured to receive the 3D surface information via the one of the one or more wireless full-duplex communication channels, and render the 3D surface information into an interactive 3D graphical representation compatible to a web browser.

The rendering of the 3D surface information into an interactive 3D graphical representation may be performed in real time, such that the displaying unit may be configured to display the rendered 3D surface information in real time.

Thus, the intraoral scanning system may enable processing of the plurality of 2D scan images to provide or generate the 3D surface information in the handheld intraoral scanning device, thereby eliminating a use of additional infrastructure, such as a computing device. The intraoral scanning system may further enable the one or more client devices to directly communicate with the handheld intraoral scanning device to receive the 3D surface information and render the interactive 3D graphical representation to be accessed by a user.

In some embodiments, the web server interface and the one or more client devices may be connected to a common web network. The intraoral scanning system enables the web server interface of the handheld intraoral scanning device and the one or more client devices to connect to the one (or common) of the one or more wireless full-duplex communication channels.

In some embodiments, multiple of the one or more client devices may be configured to receive the 3D surface information via a wireless full-duplex communication channel of the one or more wireless full-duplex communication channels. The multiple of the one or more client devices may further render the 3D surface information into the interactive 3D graphical representation compatible to the web browser. Thus, the intraoral scanning system enables access of the interactive 3D graphical representation to different users at a same time.

In some embodiments, the one or more client devices may be at least one of a displaying unit, a tablet, or a smartphone. The one or more client devices includes a capability to render the interactive 3D graphical representation for the user.

In some embodiments, the one or more client devices may be a computer. The computer may be, for example, a computing device with a significant processing capacity.

In some embodiments, a bandwidth of the one of the one or more wireless full-duplex communication channels is monitored by a monitoring unit of the intraoral scanning system (102). When the bandwidth is below a minimum bandwidth, the handheld intraoral scanning device may be configured to down-sample the 3D surface information to be transmitted via the one of the one or more wireless full-duplex communication channels. Thus, the intraoral scanning system enables the transmission of the 3D surface information without a lag in real-time or near real-time.

In some embodiments, the bandwidth of the one of the one or more wireless full-duplex communication channels is monitored by the monitoring unit of the intraoral scanning system. The handheld intraoral scanning device may further include a temporary storage unit configured to store the 3D surface information, when the bandwidth of the one of the one or more wireless full-duplex communication channels is determined to be below the minimum bandwidth. The temporary storage unit may transmit the stored 3D surface information, when the bandwidth is determined to be above or equal to the minimum bandwidth. In case the transmission of the 3D surface information is unrequired in the real-time, the intraoral scanning system enables transmission of the stored 3D surface information when the bandwidth is determined to be above or equal the minimum bandwidth.

In some embodiments, when the bandwidth of the one of the one or more wireless full-duplex communications is below the minimum bandwidth longer than a maximum period, the handheld intraoral scanning device may be configured to compress and store the 3D surface information into a memory unit of the handheld intraoral scanning device. The intraoral scanning system enables compression of the 3D surface information to save storage space in the memory unit. The stored 3D surface information may be utilized at later by the user, when the bandwidth is determined to be above or equal the minimum bandwidth.

In some embodiments, the monitoring unit may be configured to determine when a connection to the one of the one or more wireless full-duplex communication channels is lost. The handheld intraoral scanning device may be configured to compress and store the 3D surface information into the memory unit of the handheld intraoral scanning device, based on the determination that the connection is lost. The intraoral scanning system enables storage of the 3D surface information when the connection is lost, to securely store the provided 3D surface information based on the captured plurality of 2D scan images.

In some embodiments, the handheld intraoral scanning device may be configured to transmit via a wireless communication interface of the intraoral scanning system, the stored 3D surface information in the memory unit when the scanning session is finished. The intraoral scanning system enables storage of the 3D surface information in the memory unit of the handheld intraoral scanning device, that may be utilized by the user any time after the scanning session is finished.

In some embodiments, the handheld intraoral scanning device further includes the monitoring unit that is configured to transmit a status input based on at least one of the determination that the bandwidth is less than the minimum bandwidth, or the determination that the connection to the one of the one or more wireless full-duplex communication channels is lost. The handheld intraoral scanning device further includes a scanning feedback unit configured to receive the status input from the monitoring unit, and provide a scanning feedback signal to the user of the handheld intraoral scanning device, while receiving the status input. The scanning feedback signal may be configured to provide guidance to the user to an area of the dental arch where a scanning quality of the scanning session is low and unable to provide the 3D surface information. The feedback signal provided by the intraoral scanning system may be used to guide the user to capture more number of the plurality of 2D scan images for the generation of the 3D surface information accurately.

In some embodiments, the scanning feedback signal may include an acoustic feedback signal configured to guide the user towards the area of the dental arch. The acoustic feedback signal may be, for example, sounds that may be utilized by the user.

In some embodiments, the scanning feedback signal may include at least one of haptic feedback, or light emitted by a plurality of light emitting diodes of the handheld intraoral scanning device. The intraoral scanning system may provide the haptic feedback and visual feedback in form of the light, that may be utilized by the user to capture a greater number of the plurality of 2D scan images.

In some embodiments, the handheld intraoral scanning device may include a vibrator configured to provide the haptic feedback. An increase in the vibration may indicate an increasing distance between the area of the dental arch and the handheld intraoral scanning device. A decrease in the vibration may indicate a decreasing distance between the area of the dental arch and the handheld intraoral scanning device. Thus, the intraoral scanning system enables determination of the area of the dental arch from which a greater number of the plurality of 2D scan images are required.

In some embodiments, the plurality of light emitting diodes is divided into a left group of light emitting diodes and a right group of light emitting diodes. The left group and the right group are configured to emit a flash of light when the handheld intraoral scanning device is arranged at right or left to the area of the dental arch respectively. The plurality of light emitting diodes arranged in such a manner enables usage of the handheld intraoral scanning device efficiently without having to repeatedly rotate the handheld intraoral scanning device while scanning.

In some embodiments, the handheld intraoral scanning device may be configured to broadcast the 3D surface information to multiple of the one or more client devices connected to the handheld intraoral scanning device via the one of the one or more wireless full-duplex communication channels. Thus, the intraoral scanning system enables utilization of the multiple of the one or more client devices simultaneously.

In some embodiments, the handheld intraoral scanning device may be configured to generate a 3D model representation of the dental arch by combining a plurality of the 3D surface information provided by the handheld intraoral scanning device. The handheld intraoral scanning device may be configured to transmit the 3D model via the one of the one or more wireless full-duplex communication channels. Thus, the intraoral scanning system enables generation of the 3D model in the handheld intraoral scanning device, without having to use the external infrastructure, such as the computing device.

In another aspect, the present disclosure provides a method for the intraoral scan registration. The method may include capturing, by a handheld intraoral scanning device, a plurality of two-dimensional (2D) scan images during a scanning session of a dental arch. The method may further include providing, by the handheld intraoral scanning device, three-dimensional (3D) surface information based on the plurality of 2D scan images captured during the scanning session. The method may further include establishing, by one or more client devices, a connection to one of one or more wireless full-duplex communication channels by forwarding an identification number to the handheld intraoral scanning device via a web network. The method may further include receiving, by the one or more client devices, the 3D surface information via the one of the one or more wireless full-duplex communication channels. The method may further include rendering, on the one or more client devices, the 3D surface information into an interactive 3D graphical representation compatible to a web browser.

In yet another aspect, the present disclosure provides an intraoral scanning system for generation of a three-dimensional (3D) model and rendering an interactive 3D graphical representation based on the 3D model. The intraoral scanning system may include a handheld intraoral scanning device configured to capture a first plurality of two-dimensional (2D) scan images and a second plurality of 2D scan images containing surface information of a patient's dental arch during a first time frame and a second time frame, respectively. The first time frame is before the second time frame. The handheld intraoral scanning device may be further configured to process the first plurality of 2D scan images and the second plurality of 2D scan images into first 3D surface information and second 3D surface information, respectively. The handheld intraoral scanning device may be further configured to generate a first 3D scan patch and a second 3D scan patch by transforming the first 3D surface information into first real-world 3D coordinates and first texture information, and transforming the second 3D surface information into second real-world 3D coordinates and second texture information by using calibration data stored on a memory unit. The handheld intraoral scanning device may be further configured to register the second 3D scan patch to at least the first 3D scan patch by locating corresponding data points between the first 3D scan patch and the second 3D scan patch. The handheld intraoral scanning device may be further configured to fuse the first 3D scan patch and the second 3D scan patch together to form a 3D model. The handheld intraoral scanning device may be further configured to store the first texture information and the second texture information together with the formed 3D model. The handheld intraoral scanning device comprises a web server interface configured to communicate via a web network and establish a connection to one or more wireless full-duplex communication channels. The intraoral scanning system may further include one or more client devices. Each of the one or more client devices may be configured to establish a connection to one of the one or more wireless full-duplex communication channels by forwarding an identification number to the handheld intraoral scanning device via the web network. Each of the one or more client devices may be further configured to receive the 3D model via the one of the one or more wireless full-duplex communication channels. Each of the one or more client devices may be further configured to render the 3D model into an interactive 3D graphical representation compatible to a web browser.

In another aspect, the present disclosure provides a method for generation of a three-dimensional (3D) model and rendering an interactive 3D graphical representation based on the 3D model. The method may include capturing, by a handheld intraoral scanning device, a first plurality of two-dimensional (2D) scan images and a second plurality of 2D scan images containing surface information of a patient's dental arch during a first time frame and a second time frame, respectively. The first time frame is before the second time frame. The method may further include processing, by the handheld intraoral scanning device, the first plurality of 2D scan images and the second plurality of 2D scan images into first three-dimensional (3D) surface information and second 3D surface information, respectively. The method may further include generating, by the handheld intraoral scanning device, a first 3D scan patch and a second 3D scan patch by transforming the first 3D surface information into first real-world 3D coordinates and first texture information, and transforming the second 3D surface information into second real-world 3D coordinates and second texture information by using calibration data. The method may further include registering, by the handheld intraoral scanning device, the second 3D scan patch to at least the first 3D scan patch by locating corresponding data points between the first 3D scan patch and the second 3D scan patch. The method may further include fusing, by the handheld intraoral scanning device, the first 3D scan patch and the second 3D scan patch together to form a 3D model. The method may further include storing, by the handheld intraoral scanning device, the first texture information and the second texture information together with the formed 3D model. The method may further include establishing, by one or more client devices, a connection to one of one or more wireless full-duplex communication channels by forwarding an identification number to the handheld intraoral scanning device via a web network. The method may further include receiving, by the one or more client devices, the 3D model via the one of the one or more wireless full-duplex communication channels. The method may further include rendering, on the one or more client devices, the 3D model into an interactive 3D graphical representation compatible to a web browser.

In yet another aspect, the present disclosure provides a computer programmable product comprising a non-transitory computer readable medium having stored thereon computer executable instructions, which when executed by a processing circuitry, cause the processing circuitry to carry out operations. The operations may include capturing, by a handheld intraoral scanning device, a plurality of two-dimensional (2D) scan images during a scanning session of a dental arch. The operations may further include providing, by the handheld intraoral scanning device, three-dimensional (3D) surface information based on the plurality of 2D scan images captured during the scanning session. The operations may further include establishing, by one or more client devices, a connection to one of one or more wireless full-duplex communication channels by forwarding an identification number to the handheld intraoral scanning device via a web network. The operations may further include receiving, by the one or more client devices, the 3D surface information via the one of the one or more wireless full-duplex communication channels. The operations may further include rendering, on the one or more client devices, the 3D surface information into an interactive 3D graphical representation compatible to a web browser.

The foregoing summary is illustrative only and is not intended to be in any way limiting. In addition to the illustrative aspects, embodiments, and features described above, further aspects, embodiments, and features will become apparent by reference to the drawings and the following detailed description.

### Effect(s) of the Invention

According to the present disclosure, an intraoral scanning system, a method and a computer programmable product are provided. One of the purposes of the present disclosure is to provide an enhanced processing capability in a handheld intraoral scanning device to provide three-dimensional (3D) surface information.

Conventional systems may include intraoral scanners that are utilized to capture two-dimensional (2D) intraoral scans of dental arches of patients. The conventional intraoral scanners may possess limited processing capability, that may only be utilized to capture the 2D intraoral scans of the dental arches. In order to generate the 3D information of the dental arches, the conventional intraoral scanners may need to rely on external infrastructure, such as a computing device (for example, a powerPC). To utilize the computing device, the conventional intraoral scanners may need to connect with the computing device via a network. In certain cases, the computing device may be at a different location than the intraoral scanner. For example, the computing device may be bulky in size and may need to be placed in a different room than a treatment room of a dental clinic. In such a case, a user such as a dentist may need to access the computing device in the other room after capturing the 2D intraoral scans. The user may need to connect the conventional intraoral scanners with the computing device using the network. The computing device, based on the 2D intraoral scans may generate the 3D information of the dental arches and render the 3D information on a display screen. Thus, usage of the conventional system for the intraoral scan registration may be time consuming and difficult for the users.

On the other hand, the intraoral scanning system of the present disclosure includes a handheld intraoral scanning device. The intraoral scanning system may provide an enhanced processing capability in a handheld intraoral scanning device. The handheld intraoral scanning device may include a web server interface configured to communicate via a web network and establish a connection to a communication network. The handheld intraoral scanning device of the present disclosure may capture a plurality of 2D scan images of the dental arches of the patient. Based on the captured plurality of 2D scan images, the handheld intraoral scanning device may provide 3D surface information of the dental arches. Therefore, the intraoral scanning system of the present disclosure may eliminate a need to external infrastructure such as the computing system to generate the 3D surface information. Furthermore, the intraoral scanning system of the present disclosure includes one or more client devices that may be communicatively coupled to the handheld intraoral scanning device via the communication network. For example, the one or more client devices may be a smartphone, a tablet or a laptop that may be kept in a same room (such as the treatment room) as that of the handheld intraoral scanning device. The one or more client devices may receive the 3D surface information and render an interactive 3D graphical representation based on the 3D surface information. Thus, a need to switch between different rooms to access the 3D surface information is further eliminated by the intraoral scanning system. Hence, the intraoral scanning system may provide a user-friendly and a time efficient process for the intraoral scan registration.

Further, in the conventional systems, when infrastructure of the network is unable to fulfil the infrastructure requirements, the scan data (such as the 2D scans) may get lost on the way or may lag when being transmitted to the computing device. Furthermore, there may be a limitation on a number of the intraoral scanners that may be connected with the network at a time in the dental clinics. Moreover, the conventional systems allow a single computing device to handle a single intraoral scanner at a time, thereby making parallel processing difficult. On the other hand, the intraoral scanning system of the present disclosure enables monitoring of a bandwidth of the communication network. In case, the bandwidth is less than a required bandwidth, the intraoral scanning system of the present disclosure may down-sample the 3D surface information before transmission to the one or more client devices. Furthermore, the intraoral scanning system enables storage of the 3D surface information in a memory unit of the handheld intraoral scanning device, after a scanning session of the dental arches is finished. Thus, the intraoral scanning system of the present disclosure eliminates a problem of loss of the scan data. The intraoral scanning system may further enable the handheld intraoral scanning device to broadcast the 3D surface information to multiple of the one or more client devices connected to the handheld intraoral scanning device via the same network at a same time. Thus, multiple users may be able to access the 3D surface information at the same time.

### BRIEF DESCRIPTION OF THE DRAWINGS

The present disclosure is illustrated by way of example and not by way of limitation in the figures of the accompanying drawings, in which the like reference numerals indicate like elements and in which:

FIG. 1 is a diagram that illustrates a network environment of an intraoral scanning system for oral scan registration, in accordance with an example embodiment;

FIG. 2 illustrates a block diagram of a handheld intraoral scanning device, in accordance with an example embodiment;

FIG. 3 illustrates a block diagram of one or more client devices, in accordance with an example embodiment;

FIG. 4 is a schematic diagram that illustrates an environment for communication of the handheld intraoral scanning and the one or more client devices, in accordance with an example embodiment;

FIG. 5 is a schematic diagram that illustrates capture of a plurality of two-dimensional (2D) scan images to generate three-dimensional (3D) surface information, in accordance with an example embodiment;

FIG. 6 is a schematic diagram that illustrates transmission of the 3D surface information and render of interactive 3D graphical representation, in accordance with an example embodiment;

FIG. 7 is a sequence diagram that depicts render of the interactive 3D graphical representation based on the transmitted 3D surface information, in accordance with an example embodiment;

FIG. 8 illustrates an example flowchart comprising different cases for transmission of the 3D surface information to the one or more client devices, in accordance with an example embodiment;

FIG. 9 illustrates an example flowchart for generation of a 3D model based on the 3D surface information, in accordance with another example embodiment;

FIG. 10 is a sequence diagram that depicts render of the interactive 3D graphical representation based on the transmitted 3D model, in accordance with an example embodiment;

FIG. 11 is a schematic diagram that depicts an exemplary environment for capture of the plurality of 2D scan images and render of the interactive 3D graphical representation in real-time, in accordance with an example embodiment; and

FIG. 12 is an illustration of an intermediate processing unit in communication with the handheld intraoral scanner and the one or more client devices.

### DETAILED DESCRIPTION OF THE INVENTION

In the following description, for purposes of explanation, numerous specific details are set forth in order to provide a thorough understanding of the present disclosure. It will be apparent, however, to one skilled in the art that the present disclosure may be practiced without these specific details. In other instances, systems and methods are shown in block diagram form only in order to avoid obscuring the present disclosure.

Reference in this specification to “one embodiment” or “an embodiment” means that a particular feature, structure, or characteristic described in connection with the embodiment is included in at least one embodiment of the present disclosure. The appearance of the phrase “in one embodiment” in various places in the specification are not necessarily all referring to the same embodiment, nor are separate or alternative embodiments mutually exclusive of other embodiments. Further, the terms “a” and “an” herein do not denote a limitation of quantity, but rather denote the presence of at least one of the referenced items. Moreover, various features are described which may be exhibited by some embodiments and not by others. Similarly, various requirements are described which may be requirements for some embodiments but not for other embodiments.

Some embodiments of the present disclosure will now be described more fully hereinafter with reference to the accompanying drawings, in which some, but not all, embodiments of the disclosure are shown. Indeed, various embodiments of the disclosure may be embodied in many different forms and should not be construed as limited to the embodiments set forth herein; rather, these embodiments are provided so that this disclosure will satisfy applicable legal requirements. Like reference numerals refer to like elements throughout. As used herein, the terms “data,” “content,” “information,” and similar terms may be used interchangeably to refer to data capable of being transmitted, received and/or stored in accordance with embodiments of the present disclosure. Further, the terms “processor”, “controller” and “processing circuitry” and similar terms may be used interchangeably to refer to the processor capable of processing information in accordance with embodiments of the present disclosure. Further, the terms “electronic equipment”, “electronic devices” and “devices” are used interchangeably to refer to electronic equipment monitored by the system in accordance with embodiments of the present disclosure. Thus, use of any such terms should not be taken to limit the spirit and scope of embodiments of the present disclosure.

The embodiments are described herein for illustrative purposes and are subject to many variations. It is understood that various omissions and substitutions of equivalents are contemplated as circumstances may suggest or render expedient but are intended to cover the application or implementation without departing from the spirit or the scope of the present disclosure. Further, it is to be understood that the phraseology and terminology employed herein are for the purpose of the description and should not be regarded as limiting. Any heading utilized within this description is for convenience only and has no legal or limiting effect.

As used in this specification and claims, the terms “for example” “for instance” and “such as”, and the verbs “comprising,” “having,” “including” and their other verb forms, when used in conjunction with a listing of one or more components or other items, are each to be construed as open ended, meaning that that the listing is not to be considered as excluding other, additional components or items. Other terms are to be construed using their broadest reasonable meaning unless they are used in a context that requires a different interpretation.

An intraoral scanning system, a method and a computer programmable product are provided for intraoral scan registration using a handheld intraoral scanning device and render of three-dimensional (3D) surface information on one or more client devices.

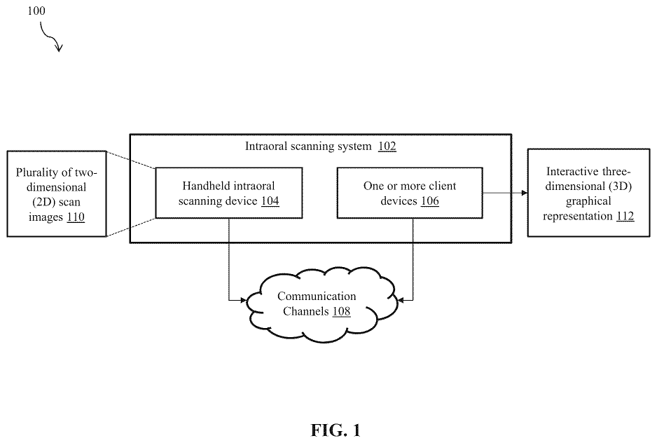

For instance, an exemplary network environment of the intraoral scanning system for oral scan registration is provided below with reference to FIG. 1.

FIG. 1 is a diagram that illustrates an exemplary network environment 100 of intraoral scanning system 102 for oral scan registration, in accordance with an example embodiment. The intraoral scanning system 102 may include a handheld intraoral scanning device 104 and one or more client devices 106. The network environment 100 may further include communication channels 108 that may be configured to communicatively couple the handheld intraoral scanning device 104 and the one or more client devices 106. The network environment 100 may further include a plurality of two-dimensional (2D) scans 110 and an interactive three-dimensional (3D) graphical representation 112. Further, it is possible that one or more components may be rearranged, changed, added, and/or removed without deviating from the scope of the present disclosure.

The intraoral scanning system 102 may be utilized for registration of intraoral scans. The intraoral scanning system 102 may include multiple devices, such as the handheld intraoral scanning device 104 and the one or more client devices 106 that may communicate with each other to register the intraoral scans.

The handheld intraoral scanning device 104 may include enhanced processing capabilities that may be required to process the plurality of 2D scan images 110. The handheld intraoral scanning device 104 may be configured to capture the plurality of 2D scan images 110 during a scanning session of a dental arch. The plurality of 2D scan images 110 may be images of the dental arch of a user, such as a patient. The plurality of 2D scan images 110 may include images of the dental arch of the patient from various angles. The plurality of 2D scan images 110 may be captured by a user, such as a dentist in the scanning session of the dental arch of the user, by use of the handheld intraoral scanning device 104.

Based on the captured plurality of 2D scan images 110, the handheld intraoral scanning device 104 may be configured to provide (or generate) 3D surface information. The 3D surface information may be a digital representation of the dental arch of the patient depicted in a 3D space. The 3D surface information may include, for example, a 3D point cloud data corresponding to the plurality of 2D scan images 110. The 3D point cloud data may correspond to 3D real-world coordinates.

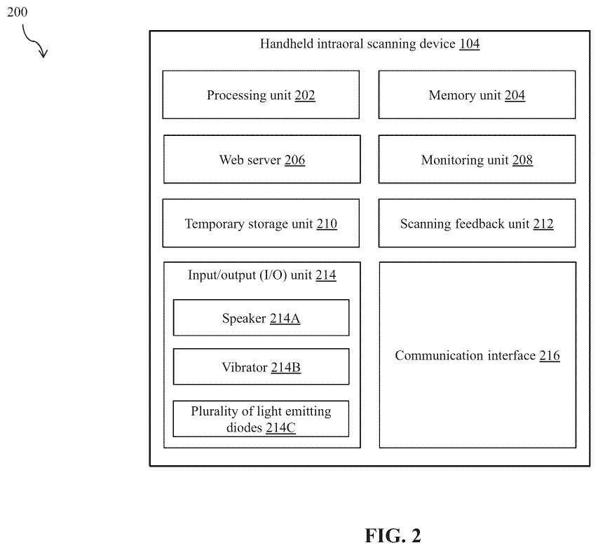

The handheld intraoral scanning device 104 may include a web server interface that may be configured to communicate via a web network and establish a connection to the communication channels 108. The handheld intraoral scanning device 104 may be configured to execute the web server interface to provide the 3D surface information based on the 2D scan images 110. The handheld intraoral scanning device 104 may further include a processing unit, a memory unit, a communication interface, and additional components. The processing unit, the memory unit, the communication interface, and the additional components may be communicatively coupled to each other. Details of the components of the handheld intraoral scanning device 104 are further provided, for example, in FIG. 2.

The one or more client devices 106 may include processing capabilities that may be required to process the 3D surface information. The one or more client devices 106 may be configured to establish a connection to one of the communication channels 108. The one or more client devices 106 may further receive the 3D surface information from the handheld intraoral scanning device 104. The one or more client devices 106 may further render the 3D surface information into an interactive 3D graphical representation 112.

The interactive 3D graphical representation 112 may be rendered on a displaying unit of the one or more client devices 106. The interactive 3D graphical representation 112 may be viewed by the users, such as the dentists on the displaying unit of the one or more client devices 106. A view of the interactive 3D graphical representation 112 may be modified by the users, based on a preference. For example, a perspective of the interactive 3D graphical representation 112 may be changed or the interactive 3D graphical representation 112 may be zoomed-in or zoomed-out as per the preference of the users. The interactive 3D graphical representation 112 may be independently rendered on the one or more client devices 106. Therefore, the interactive 3D graphical representation 112 may be accessed independently by multiple users of the respective one or more client devices 106.

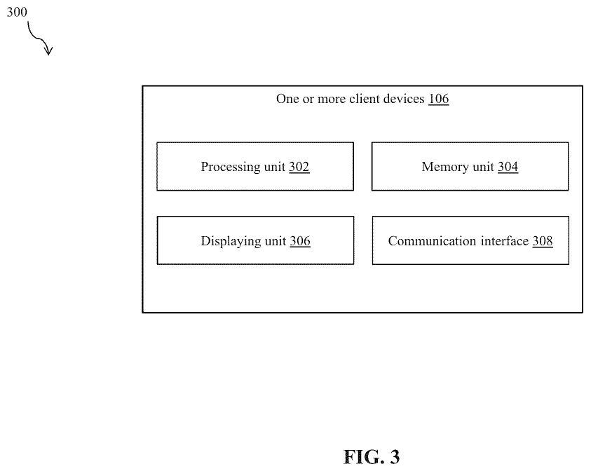

The one or more client devices 106 may be any user accessible device such as a displaying unit, a mobile phone, a smartphone, a tablet, a computer, an artificial realty (XR) device, and the like. In some examples, the displaying unit may be a part of the one or more client devices 106. The displaying unit of the one or more client devices 106 may be a touch screen display. The one or more client devices 106 may comprise the processing unit, the memory unit, and a communication interface. The processor, the memory and the communication interface may be communicatively coupled to each other. Additional, different, or fewer components may be provided. Further, it is possible that one or more components may be rearranged, changed, added, and/or removed without deviating from the scope of the present disclosure. Details of the components of the one or more client devices 106 are further provided, for example, in FIG. 3.

The communication channels 108 may be wired, wireless, or any combination of wired and wireless communication networks, such as cellular, wireless fidelity (Wi-Fi), internet, local area networks, or the like. In accordance with an embodiment, the communication channels 108 may be one or more wireless full-duplex communication channels. In one embodiment, the communication channels 108 may include one or more networks such as a data network, a wireless network, a telephony network, or any combination thereof. It is contemplated that the data network may be any local area network (LAN), metropolitan area network (MAN), wide area network (WAN), a public data network (e.g., the Internet), short range wireless network, or any other suitable packet-switched network, such as a commercially owned, proprietary packet-switched network, e.g., a proprietary cable or fiber-optic network, and the like, or any combination thereof. In addition, the wireless network may be, for example, a cellular network and may employ various technologies including enhanced data rates for global evolution (EDGE), general packet radio service (GPRS), global system for mobile communications (GSM), Internet protocol multimedia subsystem (IMS), universal mobile telecommunications system (UMTS), etc., as well as any other suitable wireless medium, e.g., worldwide interoperability for microwave access (WiMAX), Long Term Evolution (LTE) networks (for e.g. LTE-Advanced Pro), 5G New Radio networks, ITU-IMT 2020 networks, code division multiple access (CDMA), wideband code division multiple access (WCDMA), wireless fidelity (Wi-Fi), wireless LAN (WLAN), Bluetooth, Internet Protocol (IP) data casting, satellite, mobile ad-hoc network (MANET), and the like, or any combination thereof. The handheld intraoral scanning device 104 may be configured to communicate with the one or more client devices 106 via the communication channels 108.

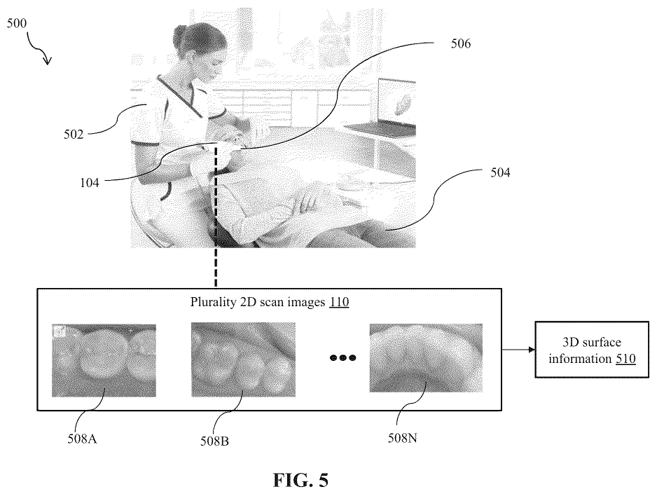

In operation, a user may require a dental treatment. In such a case, the intraoral scanning system 102 may be utilized by the dentist to provide the dental treatment to the user. In an embodiment, the user may be present at a dental clinic. In such a case, the intraoral scanning system 102 may be utilized in a treatment room of the dental clinic. In another embodiment, the user may have requested for a home visit for the dental treatment. In such a case, the intraoral scanning system 102 may be utilized in the home of the user. To start the dental treatment, the handheld intraoral scanning device 104 may be utilized by the dentist to capture the plurality of 2D scan images 110 of the dental arch of the user. Based on the captured plurality of 2D scan images 110, the handheld intraoral scanning device 104 may provide the 3D surface information. Details of the capture of the plurality of 2D scan images 110 and providing of the 3D surface information is further provided, for example, in FIG. 5.

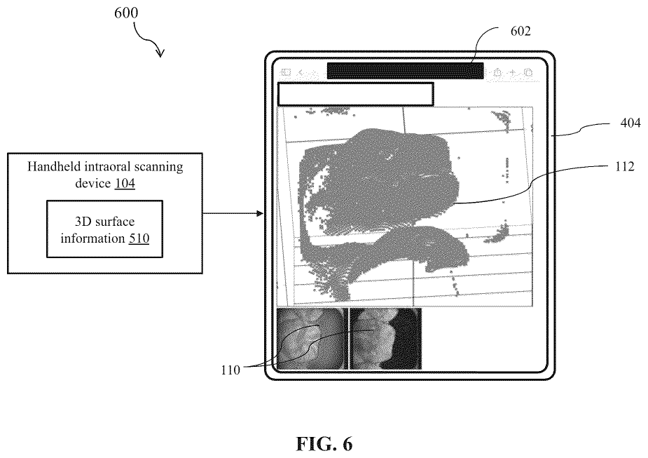

Further, after capturing the plurality of 2D scan images 110, the dentist may need to view human-readable 3D data of the plurality of 2D scan images 110 as part of the dental treatment. To view the human-readable 3D data of the plurality of 2D scan images 110, the handheld intraoral scanning device 104 and the one or more client devices 106 may need to be connected to a common communication channel of the communication channels 108. Thus, the web server interface of the handheld intraoral scanning device 104 may communicate via the web network to establish the connection with the one or more wireless full-duplex communication channels. The one or more client devices 106 may also establish the connection with one of the one or more wireless full-duplex communication channels. To establish the connection, the one or more client devices 106 may forward an identification number to the handheld intraoral scanning device 104 via the web network. Details of the connection of the one or more client devices 106 with one of the one or more wireless full-duplex communication channels are further provided, for example, in FIG. 6.

After connection of the handheld intraoral scanning device 104 and the one or more client devices 106 via the common communication channel, the one or more client devices 106 may receive the 3D surface information. For example, the dentist may utilize the tablet as one of the client device to receive the 3D surface information. The tablet may be configured to render the 3D surface information into the interactive 3D graphical representation 112 compatible to the web browser of the tablet. The interactive 3D graphical representation 112 may be utilized as the human-readable 3D data of the plurality of 2D scan images 110 by the dentist. The interactive 3D graphical representation 112 may be modified, for example, by use of gestures provided as an input by the dentist to the tablet. Details of the render of the 3D surface information into the interactive 3D graphical representation 112 are further provided, for example, in FIG. 6.

FIG. 2 illustrates a block diagram 200 of the handheld intraoral scanning device 104, in accordance with an example embodiment. FIG. 2 is explained in conjunction with elements of FIG. 1. The handheld intraoral scanning device 104 may include at least one processing unit (hereinafter, also referred to as “processing unit 202”), a memory unit 204, a web server interface 206, a monitoring unit 208, a temporary storage unit 210, a scanning feedback unit 212 an input/output (I/O) unit 214 and a communication interface 216.

The processing unit 202 may be embodied in a number of different ways. For example, the processing unit 202 may be embodied as one or more of various hardware processing means such as a coprocessor, a microprocessor, a controller, a digital signal processor (DSP), a processing element with or without an accompanying DSP, or various other processing circuitry including integrated circuits such as, for example, an ASIC (application specific integrated circuit), an FPGA (field programmable gate array), a microcontroller unit (MCU), a hardware accelerator, a special-purpose computer chip, or the like. In an embodiment, the processing unit 202 may be embodied as a high-performance microprocessor having series of System on Chip (SOCs) which includes relative powerful and power-efficient Graphics Processing Units (GPUs) and Central Processing Units (CPUs) and a small form factor. As an example, the form factor of the processing unit 202 may be 70 millimeters (mm) x 45 mm. As such, in some embodiments, the processing unit 202 may include one or more processing cores configured to perform independently. A multi-core processor may enable multiprocessing within a single physical package. Additionally, or alternatively, the processing unit 202 may include one or more processors configured in tandem via the bus to enable independent execution of instructions, pipelining and/or multithreading.

In some embodiments, the processing unit 202 may be configured to capture the plurality of 2D scan images 110 during the scanning session of the dental arch of the user, such as a patient requiring the dental treatment. The plurality of 2D scan images 110 may include images of the dental arch of the patient from various angles. Based on the plurality of 2D scan images 110, the processing unit 202 may provide the 3D surface information. For example, the 3D surface information may include, for example, the 3D point cloud data corresponding to the plurality of 2D scan images 110.

Additionally, or alternatively, the processing unit 202 may include one or more processors capable of processing large volumes of workloads and operations to provide support for big data analysis. In an example embodiment, the processing unit 202 may be in communication with the memory unit 204 via a bus for passing information among components of the handheld intraoral scanning device 104.

The memory unit 204 may be non-transitory and may include, for example, one or more volatile and/or non-volatile memories. In other words, for example, the memory unit 204 may be an electronic storage device (for example, a computer readable storage medium) comprising gates configured to store data (for example, bits) that may be retrievable by a machine (for example, a computing device like the processing unit 202). The memory unit 204 may be configured to store information, data, content, applications, instructions, or the like, for enabling the apparatus to carry out various functions in accordance with an example embodiment of the present disclosure. For example, the memory unit 204 may be configured to store the 3D surface information after the scanning session of the dental arch is finished. In certain cases, the memory unit 204 may be configured to store compressed 3D surface information, when a bandwidth of one of the communication channels 108 is below a minimum bandwidth longer than a maximum period, or a connection to one of the communication channels 108 is lost. In some embodiments, the memory unit 204 may be configured to store calibration data required to generate a 3D model corresponding to the plurality of 2D scan images. As exemplarily illustrated in FIG. 2, the memory unit 204 may be configured to store instructions for execution by the processing unit 202. As such, whether configured by hardware or software methods, or by a combination thereof, the processing unit 202 may represent an entity (for example, physically embodied in circuitry) capable of performing operations according to an embodiment of the present disclosure while configured accordingly. Thus, for example, when the processing unit 202 is embodied as the microprocessor, the processing unit 202 may be specifically configured hardware for conducting the operations described herein. Alternatively, as another example, when the processing unit 202 is embodied as an executor of software instructions, the instructions may specifically configure the processing unit 202 to perform the algorithms and/or operations described herein when the instructions are executed. The processing unit 202 may include, among other things, a clock, an arithmetic logic unit (ALU) and logic gates configured to support operation of the processing unit 202.

The web server interface 206 may be a software, a hardware or a combination thereof that may be configured to store and provide data to the web browser on the one or more client devices 106. For example, the 3D surface information may be provided to the web browser of the one or more client devices 106 via the web server interface 206. As the web server interface 206 may be accessed by any web browser, a need of installation of an additional software by the one or more client devices 106, to connect to the web server interface 206 may be eliminated. The web server interface 206 may communicate to one of the communication channels 108 via a web network. In an example, the web server interface 206 and the one or more client devices 106 may communicate to a common wireless full-duplex communication channel via the web network for transmission and reception of the 3D surface information. The web server interface 206 and the web browser may communicate via Hypertext Transfer Protocol (HTTP), Simple Mail Transfer Protocol (SMTP), or File Transfer Protocol (FTP). Once the web server interface 206 and the web browser are connected, the web server interface 206 may provide a web application on the web browser. Details of the connection of the web server interface 206 and the web browser are further provided, for example, in FIG. 6.

The monitoring unit 208 may be a software, a hardware or a combination thereof that may be configured to monitor a bandwidth of one of the communication channels 108 (such as the wireless full-duplex communication channel) via which the handheld intraoral scanning device 104 and the one or more client devices 106 may be connected. Moreover, the monitoring unit 208 may be configured to monitor a connection of one of the communication channels 108 via which the handheld intraoral scanning device 104 and the one or more client devices 106 may be connected. Furthermore, the monitoring unit 208 may be configured to transmit a status input to the one or more client devices 106, based on the monitored bandwidth and the connection.

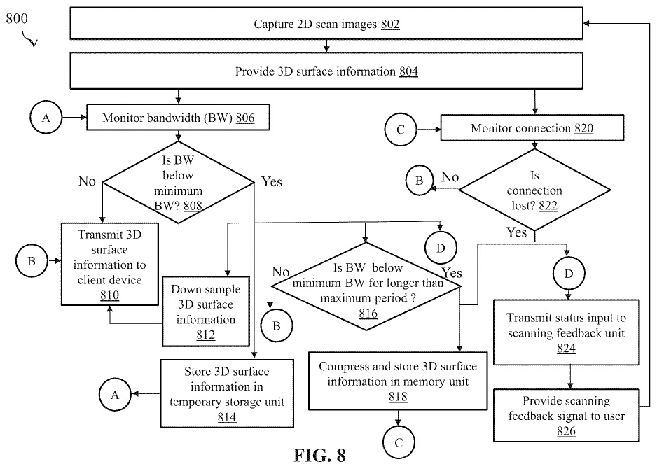

In an embodiment, based on the monitoring, the monitoring unit 208 may determine that the bandwidth is below a minimum bandwidth. In such a case, the monitoring unit 208 may provide information that the bandwidth is below the minimum bandwidth to the processing unit 202. The processing unit 202 may down-sample the 3D surface information, based on the received information. In another embodiment, based on the monitoring, the monitoring unit 208 may determine that the bandwidth is below the minimum bandwidth for longer than a maximum period. In such a case, the processing unit 202 may compress and store the 3D surface information into the memory unit 204. In some embodiments, the based on the monitoring, the monitoring unit 208 may determine that the connection between the handheld intraoral scanning device 104 and the one or more client devices 106 is lost. In such a case, the processing unit 202 may compress and store the 3D surface information into the memory unit 204. Details of the monitoring of the bandwidth and the connection, and transmission of the status input are further provided, for example, in FIG. 8.

The temporary storage unit 210 may be a software, a hardware or a combination thereof that may be configured to store the 3D surface information when the bandwidth of the one of the communication channels 108 (such as the wireless full-duplex communication channel) is determined to be below the minimum bandwidth. The temporary storage unit 210 may further transmit the stored 3D surface information to the one or more client devices 106 when the bandwidth is determined to be above or equal the minimum bandwidth. Examples of the temporary storage unit 210 may include, but may not be limited to, a random access memory (RAM), or a cache memory.

The scanning feedback unit 212 may be a software, a hardware or a combination thereof that may be configured to receive the status input from the monitoring unit. Based on the received status input, the scanning feedback unit 212 may provide a scanning feedback signal to the user of the handheld intraoral scanning device 104. In an embodiment, the scanning feedback signal is used to provide guidance to the user to an area of the dental arch where a scanning quality of the scanning session is low and unable to provide the 3D surface information. For example, the scanning feedback signal may be utilized to provide an acoustic feedback signal, a haptic feedback, or a visual feedback. Details of providing the scanning feedback signal are further provided, for example, in FIG. 8.

The I/O unit 214 may include circuitry and/or software that may be configured to provide output to the user of the handheld intraoral scanning device 104. The I/O unit 214 may include a speaker 214A, a vibrator 214B, and a plurality of emitting diodes (LEDs) 214C. In an embodiment, the speaker 214A may be configured to output the acoustic feedback signal to guide the user. The vibrator 214B may be for example, a transducer configured to convert the scanning feedback signal that may be an electrical signal into a mechanical output, such as the haptic feedback in form of vibrations to guide the user. The plurality of LEDs 214C may be configured to output the scanning feedback signal in form of light to guide the user. For example, the plurality of LEDs 214C may be divided into a left group of LEDs and a right group of LEDs to emit a flash of light. Details of the acoustic feedback signal, the haptic feedback, and the visual feedback such as light are further provided, for example, in FIG. 8.

The communication interface 216 may comprise input interface and output interface for supporting communications to and from the handheld intraoral scanning device 104. The communication interface 216 may be a device or circuitry embodied in either hardware or a combination of hardware and software that is configured to receive and/or transmit data to/from the handheld intraoral scanning device 104. In this regard, the communication interface 216 may include, for example, an antenna (or multiple antennae) and supporting hardware and/or software for enabling communications with a wireless communication network. Additionally, or alternatively, the communication interface 216 may include the circuitry for interacting with the antenna(s) to cause transmission of signals via the antenna(s) or to handle receipt of signals received via the antenna(s). In some environments, the communication interface 216 may alternatively or additionally support wired communication. As such, for example, the communication interface 216 may include a communication modem and/or other hardware and/or software for supporting communication via cable, digital subscriber line (DSL), universal serial bus (USB) or other mechanisms.

FIG. 3 illustrates a block diagram 300 of the one or more client devices 106, in accordance with an example embodiment. FIG. 3 is explained in conjunction with elements of FIG. 1 and FIG. 2. The one or more client devices 106 may include a processing unit 302, a memory unit 304, a displaying unit 306 and a communication interface 308.

The processing unit 302 may be embodied in a number of different ways. For example, the processing unit 302 may be embodied as one or more of various hardware processing means such as the coprocessor, the microprocessor, the controller, the DSP, the processing element with or without the accompanying DSP, or various other processing circuitry including integrated circuits such as, for example, the ASIC, the MCU, the hardware accelerator, the special-purpose computer chip, or the like. As such, in some embodiments, the processing unit 302 may include one or more processing cores configured to perform independently. A multi-core processor may enable multiprocessing within a single physical package. Additionally, or alternatively, the processing unit 302 may include one or more processors configured in tandem via the bus to enable independent execution of instructions, pipelining and/or multithreading.

In some embodiments, the processing unit 302 may include processing capabilities that may be required to process the 3D surface information. The processing unit 302 may be configured to establish the connection to one of the communication channels 108. The processing unit 302 may further receive the 3D surface information from the handheld intraoral scanning device 104. The processing unit 302 may further render the 3D surface information into the interactive 3D graphical representation 112. Additionally, or alternatively, the processing unit 302 may include one or more processors capable of processing large volumes of workloads and operations to provide support for big data analysis. In an example embodiment, the processing unit 302 may be in communication with the memory unit 304 via a bus for passing information among components of the one or more client devices 106.

The memory unit 304 may be the non-transitory and may include, for example, the one or more volatile and/or the non-volatile memories. In other words, for example, the memory unit 304 may be the electronic storage device (for example, a computer readable storage medium) comprising gates configured to store data (for example, bits) that may be retrievable by a machine (for example, a computing device like the processing unit 302). The memory unit 304 may be configured to store information, data, content, applications, instructions, or the like, for enabling the apparatus to carry out various functions in accordance with an example embodiment of the present disclosure. For example, the memory unit 304 may be configured to store the received 3D surface information. In some embodiments, the memory unit 304 may be configured to store the interactive 3D graphical representation 112. In an embodiment, the memory unit 304 may be configured to store a 3D model received form the handheld intraoral scanning device 104.

As exemplarily illustrated in FIG. 3, the memory unit 304 may be configured to store instructions for execution by the processing unit 302. As such, whether configured by hardware or software methods, or by a combination thereof, the processing unit 302 may represent an entity (for example, physically embodied in circuitry) capable of performing operations according to an embodiment of the present disclosure while configured accordingly. Thus, for example, when the processing unit 302 is embodied as the microprocessor, the processing unit 302 may be specifically configured hardware for conducting the operations described herein. Alternatively, as another example, when the processing unit 302 is embodied as an executor of software instructions, the instructions may specifically configure the processing unit 302 to perform the algorithms and/or operations described herein when the instructions are executed. The processing unit 302 may include, among other things, the clock, the ALU and logic gates configured to support operation of the processing unit 302.

The displaying unit 306 may be configured to display the web browser, the web application and the interactive 3D graphical representation 112. In some embodiments, the displaying unit 306 may be externally connected to the one or more client devices 106. Examples of the displaying unit 306 may include, but are not limited to, a liquid crystal display (LCD), a light emitting diode (LED) display, an electroluminescent (ELD) display, a plasma display, or a cathode ray tube (CRT) display. In an embodiment, the displaying unit 306 may be a touch screen display. The displaying unit 306 may receive input in form of gestures from the user, control the interactive 3D graphical representation 112.

The communication interface 308 may comprise input interface and output interface for supporting communications to and from the one or more client devices 106. The communication interface 308 may be a device or circuitry embodied in either hardware or a combination of hardware and software that is configured to receive and/or transmit data to/from the one or more client devices 106. In this regard, the communication interface 308 may include, for example, an antenna (or multiple antennae) and supporting hardware and/or software for enabling communications with a wireless communication network. Additionally, or alternatively, the communication interface 308 may include the circuitry for interacting with the antenna(s) to cause transmission of signals via the antenna(s) or to handle receipt of signals received via the antenna(s). In some environments, the communication interface 308 may alternatively or additionally support wired communication. As such, for example, the communication interface 308 may include a communication modem and/or other hardware and/or software for supporting communication via the cable, the DSL, the USB or other mechanisms.

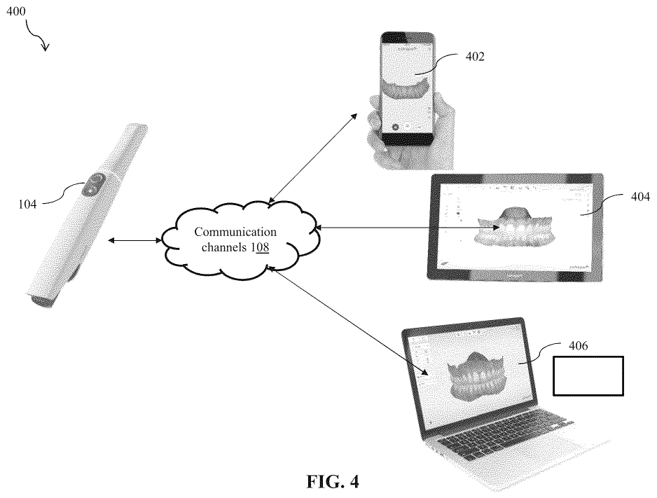

FIG. 4 is a schematic diagram 400 that illustrates an environment for communication of the handheld intraoral scanning device 104 and the one or more client devices 106, in accordance with an example embodiment. FIG. 4 is explained in conjunction with elements of FIG. 1, FIG. 2 and FIG. 3. The schematic diagram 400 may include the handheld intraoral scanning 104 and the communication channels 108. The schematic diagram 400 may further include the one or more client devices 106.

The one or more client devices 106 may include a client device 402, a client device 404, and a client device 406. In accordance with an embodiment, the one or more client devices 106 may be at least one of the displaying unit, the tablet, or the smartphone. For example, the client device 402 may be the smartphone of the user, such as the dentist. In another example, the client device 404 may be the tablet. In some embodiments, the one or more client devices 106 may be the computer. For example, the client device 406 may be the computer having the enhanced processing capabilities (for example, a PowerPC). The client device 402, the client device 404 and the client device 406 may be utilized by one or more users to view the interactive 3D graphical representation 112.

In some embodiments, the web server interface of the handheld intraoral scanning device 104 and the one or more client devices 106 may be connected to the common web network. The web network may provide access to different web pages and web applications. The common web network may be required to transfer data, such as the 3D surface information via a web application accessible via the web browser of the one or more client devices 106. In an example, the web network may be directly hosted on the handheld intraoral scanning device 104, and the one or more client devices 106 may be connected to the web network directly hosted on the handheld intraoral scanning device 104.

Once the handheld intraoral scanning device 104 and the one or more client devices 106 are connected to the common web network, the one or more client devices 106 may be configured to connect to the one of the one or more wireless full-duplex communication channels by forwarding an identification number to the handheld intraoral scanning device 104. For example, the identification number may be a unique serial number of the handheld intraoral scanning device 104 that may be entered by the user (e.g., the dentist) of a client device, such as the client device 402 via the web browser on the client device 402. The web browser may be rendered on the one or more client devices 106 based on reception of an input from the user. For instance, the user may select the web browser on the one or more client devices 106 to render the web browser.

In an example, a multicast domain name system (mDNS) protocol may be utilized to resolve a hostname (such as the identification number) to an internet protocol (IP) address to access the web server interface on the handheld intraoral scanning device 104. In some embodiments, an alias name may be provided for the identification number of the handheld intraoral scanning device 104 that may be entered by the user on the web browser to connect to the web server interface. Similarly, multiple client devices, such as the client device 404 and the client device 406 may further be connected to the web server interface of the handheld intraoral scanning device 104, based on reception of the identification number by respective users of the multiple client devices.

Once the one or more client devices 106 are connected to the web server interface of the handheld intraoral scanning device 104, the handheld intraoral scanning device 104 may provide a web application to the users of the one or more client devices 106, enabling interaction with the handheld intraoral scanning device 104 and providing a view for rendering of the interactive 3D graphical representation 112. In an example, the web server interface of the handheld intraoral scanning device 104 and the web application of the one or more client devices 106 may communicate using different communication protocols, such as a WebSocket protocol. In some embodiments, the web application may be a based on different web communication languages, such as HyperText Markup Language (HTML), JavaScript and WebAssembly. The WebAssembly module of the web application may enable compiled code for accessing the web server interface to be run on the one or more client devices 106 at a near-native speed and thus, may reduce requirements of resources needed on the one or more client devices 106.

The scanning session may be initiated by the user, such as the dentist once the handheld intraoral scanning device 104 and the one or more client devices 106 are connected, and the web application is rendered via the web browser on the one or more client devices 106. The captured plurality of 2D scan images 110 may be utilized to generate the 3D surface information by the handheld intraoral scanning device 104. Details of the capturing of the plurality of 2D scan images 110 and the generation of the 3D surface information, are further provided, for example, in FIG. 5.

FIG. 5 is a schematic diagram 500 that illustrates capture of the plurality of 2D scan images 110 to generate the 3D surface information, in accordance with an example embodiment. FIG. 5 is explained in conjunction with elements of FIG. 1, FIG. 2, FIG. 3 and FIG. 4. The schematic diagram 500 may include a user, such as a dentist 502 and a patient 504. The handheld intraoral scanning device 104 may be utilized by the dentist 502 to capture the plurality of 2D scan images 110 of a dental arch 506 of the patient 504.

In an exemplary scenario, the dentist 502 and the patient 504 may be present at the dental clinic. The scanning session of the dental arch 506 of the patient 504 may be initiated by the dentist 502 to start the dental treatment of the patient 504. The handheld intraoral scanning device 104 may include an in-built camera. The in-built camera of the handheld intraoral scanning device 104 may be placed inside a mouth of the patient 504 and moved around teeth and gums of the patient 504 to record oral topography of the patient 504. In an example, the handheld intraoral scanning device 104 may record a size and a shape of each tooth, an interdental separation, an appearance of a surface of a palate, gums, implants, prostheses, and other elements that make up an interior of an oral cavity of the patient 504. In an embodiment, the in-built camera of the handheld intraoral scanning device 104 may be moved multiple times over the teeth and the gums of the patient 504 to capture the plurality of 2D scan images 110. For example, the plurality of 2D scan images 110 may include a scan image 508A, a scan image 508B and a scan image 508N. The scan image 508A, the scan image 508B and the scan image 508N may be utilized to generate 3D surface information 510.

Once the scanning session starts, and the plurality of 2D scan images 110 may be captured, the handheld intraoral scanning device 104 may be configured to process the plurality of 2D scan images 110 by applying calibration parameters and filtering noise from the plurality of 2D scan images 110. For example, the calibration parameters of the in-built camera may be applied to process the plurality of 2D scan images 110. Moreover, the noise may be filtered from the plurality of 2D scan images 110. Based on the processing of the plurality of 2D scan images 110, the handheld intraoral scanning device 104 may be configured to provide the 3D surface information 510. In some embodiments, the 3D surface information 510 may be 3D point cloud data. The 3D surface information 510 may be transmitted to the one or more client devices 106 connected to the handheld intraoral scanning device 104. Details of the transmission of the 3D surface information 510 to the one or more client devices 106 are further provided, for example, in FIG. 6.

FIG. 6 is a schematic diagram 600 that illustrates transmission of the 3D surface information 510 and render of interactive 3D graphical representation, in accordance with an example embodiment. FIG. 6 is explained in conjunction with elements of FIG. 1, FIG. 2, FIG. 3, FIG. 4 and FIG. 5. The schematic diagram 600 may include the handheld intraoral scanning device 104 and the client device 404, such as the tablet. The handheld intraoral scanning device 104 may include the generated 3D surface information 510. The client device 404 may render the interactive 3D graphical representation 112.

In some embodiments, the handheld intraoral scanning device 104 may be configured to broadcast the generated 3D surface information 510 to multiple of the one or more client devices 106 connected to the handheld intraoral scanning device 104 via the one of the one or more wireless full-duplex communication channels. In an example, the client device 404 may receive the identification number from the user (such as the dentist) to connect to the web server interface. For example, the user may input the identification number on a web browser 602 of the client device 404. The client device 404 may forward the identification number to the handheld intraoral scanning device 104 to connect to the web server interface. Based on the connection, the handheld intraoral scanning device 104 may broadcast the 3D surface information 510 to the client device 404.

Similarly, the client device 402 and the client device 406 may be connected to the web server interface. In such a case, the handheld intraoral scanning device 104 may broadcast the 3D surface information to the client device 402 and the client device 406 (shown in FIG. 4). In an embodiment, the handheld intraoral scanning device 104 may broadcast the 3D surface information to the multiple of the one or more client devices 106 by using different communication protocols, such as WebSockets.

In some embodiments, the multiple of the one or more client devices 106 may be configured to receive independently the 3D surface information 510 via the one of the one or more wireless full-duplex communication channels. The broadcast of the 3D surface information 510 via the WebSockets may enable the reception of the 3D surface information 510 by the one or more client devices 106 independently. The 3D surface information 510 may be received independently in real-time, i.e., a speed of reception of the 3D surface information 510 may be near-instantaneous, or the 3D surface information 510 may be received independently in near real-time, i.e., the 3D surface information 510 may be received with a short delay. The multiple of the one or more client devices 106 may further render independently the 3D surface information 510 into the interactive 3D graphical representation 112 compatible to the web browser. For example, the client device 404 may render independently the 3D surface information 510 into the interactive 3D graphical representation 112 compatible to the web browser 602. In an example, the interactive 3D graphical representation 112 may be rendered on a web application running on the web browser 602.

In an embodiment, the WebSockets may enable the render of the 3D surface information 510 into the interactive 3D graphical representation 112 independently on the multiple of the one or more client devices 106. In some embodiments, the render of the 3D surface information 510 into the interactive 3D graphical representation 112 independently, may be performed by using WebGL JavaScript application program interface (API). The WebGL JavaScript API may be utilized for rendering of high-performance interactive 3D graphical representation 112 on the web application running on the web browser 602.

In an example, the interactive 3D graphical representation 112 may be rendered independently on the client device 402 for the user of the client device 402. The interactive 3D graphical representation may be rendered independently on the client device 404 for the user of the client device 404. Each of the one or more client devices 106 may have separate sessions with the web server interface of the handheld intraoral scanning device 104. Each of the one or more client devices 106 may receive a copy of the 3D surface information 510 from the web server interface. Thus, the interactive 3D graphical representation 112 may be accessed independently by different users. In certain cases, a single user may utilize multiple of the one or more client devices 106 to view the interactive 3D graphical representation 112 from different angles.

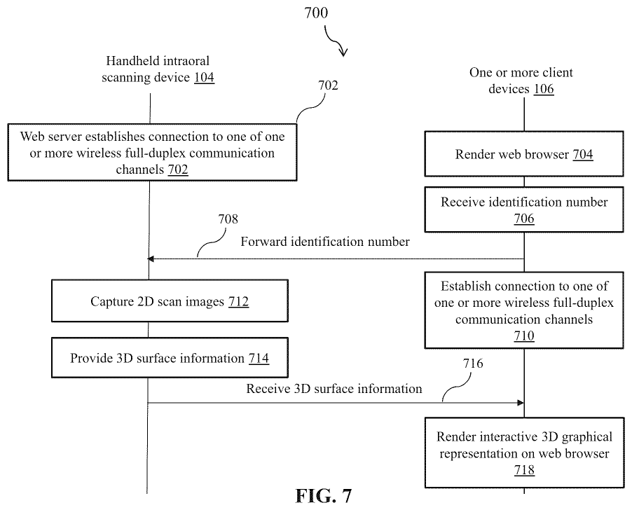

The interactive 3D graphical representation 112 may be interacted with by the users. For example, the interactive 3D graphical representation 112 may be rotated, zoomed-in, zoomed-out, and so forth as required by the users. In an embodiment, the interactive 3D graphical representation 112 may be the 3D point cloud data. In some embodiments, the interactive 3D graphical representation 112 may be a 3D model of the dental arch 506 of the patient 504. An end-to-end process of render of the interactive 3D graphical representation based on the transmitted 3D surface information is further explained in FIG. 7.

FIG. 7 is a sequence diagram 700 that depicts render of the interactive 3D graphical representation based on the transmitted 3D surface information, in accordance with an example embodiment. FIG. 7 is explained in conjunction with elements of FIG. 1, FIG. 2, FIG. 3, FIG. 4, FIG. 5 and FIG. 6. The sequence diagram 700 may include the handheld intraoral scanning device 104 and the one or more client devices 106. The sequence diagram 700 may depict operations performed by at least one of the handheld intraoral scanning device 104 and the one or more client devices 106.

At step 702, the web server interface of the handheld intraoral scanning device 104 may establish the connection with one of the communication channels 108. For example, the web server interface of the handheld intraoral scanning device 104 may communicate via the web network to establish the connection with the one or more wireless full-duplex communication channels. Details of the connection of the web server interface with the one or more wireless full-duplex communication channels are further provided, for example, in FIG. 2 and FIG. 4.

At step 704, the web browser 602 may be rendered on the one or more client devices 106. For example, the web browser 602 may be rendered on the client device 404, such as the tablet. In an exemplary scenario, the dentist 502 may have access to the client device 404. The client device 404 may be operated by the dentist 502 to access the web browser 602 on the client device 404. Details of the render of the web browser 602 are further provided, for example, in FIG. 4.

At step 706, the identification number may be received. The identification number may be received via the rendered web browser on the one or more client devices 106, such as the client device 404. For example, the dentist 502 may provide the identification number the as input on the web browser of the client device 404. Details of the reception of the identification number are further provided, for example, in FIG. 4.

At 708, the identification number may be forward to the handheld intraoral scanning device 104. The one or more client devices 106 may forward the identification number to the handheld intraoral scanning device 104 input by the user, such as the dentist 502. The identification number may be utilized to establish the connection with the web server interface of the handheld intraoral scanning device 104. Details of the forward of the identification number are further provided, for example, in FIG. 4.

At 710, the connection to the one of the one or more wireless full-duplex communication channels may be established. Based on the forwarded identification number via the web network, the connection of the one or more client devices 106 to the one of the one or more wireless full-duplex communication channels may be established. Details of the connection to the one of the one or more wireless full-duplex communication channels are further provided, for example, in FIG. 4.

At 712, the plurality of 2D scan images 110 may be captured. The handheld intraoral scanning device 104 may be configured to capture the plurality of 2D scan images 110 of the dental arch 506 of the patient 504 during the scanning session. Details of the capture of the plurality of 2D scan images 110 are further provided, for example, in FIG. 5.

At 714, the 3D surface information 510 may be provided. The handheld intraoral scanning device 104 may be configured to provide the 3D surface information 510 based on the captured plurality of 2D scan images 110. For example, the 3D surface information 510 may be the 3D point cloud data. Details of providing the 3D surface information 510 are further provided, for example, in FIG. 5.

At 716, the 3D surface information may be received. The one or more client devices 106 may be configured to receive the 3D surface information 510 from the intraoral scanning device 104. The 3D surface information 510 may be received by the one or more client devices 106 by use of the one of the communication channels 108. Details of reception of the 3D surface information 510 by the one or more client devices 106 are further provided, for example, in FIG. 5.

At 718, the interactive 3D graphical representation 112 may be rendered. The one or more client devices 106 may be configured to render the interactive 3D graphical representation 112 based on the 3D surface information 510. In an embodiment, the one or more client devices 106 may render the interactive 3D graphical representation 112 independently. Details of render of the interactive 3D graphical representation 112 are further provided, for example, in FIG. 6.

It will be understood that each step of the sequence diagram 700 may be implemented by various means, such as hardware, firmware, processor, circuitry, and/or other communication devices associated with execution of software including one or more computer program instructions. For example, one or more of the steps described above may be embodied by computer program instructions. In this regard, the computer program instructions which embody the steps described above may be stored by the memory 204 of the handheld intraoral scanning device 104, employing an embodiment of the present disclosure. As will be appreciated, any such computer program instructions may be loaded onto a computer or other programmable apparatus (for example, hardware) to produce a machine, such that the resulting computer or other programmable apparatus implements the functions specified in the sequence diagram 700. These computer program instructions may also be stored in a computer-readable memory that may direct a computer or other programmable apparatus to function in a particular manner, such that the instructions stored in the computer-readable memory produce an article of manufacture the execution of which implements the function specified in the sequence diagram 700. The computer program instructions may also be loaded onto a computer or other programmable apparatus to cause a series of operations to be performed on the computer or other programmable apparatus to produce a computer-implemented process such that the instructions which execute on the computer or other programmable apparatus provide operations for implementing the functions specified in the sequence diagram 700.

Accordingly, the steps of the sequence diagram 700 support combinations of means for performing the specified functions and combinations of operations for performing the specified functions for performing the specified functions. It will also be understood that one or more steps of the sequence diagram 700, and combinations of steps in the sequence diagram 700, may be implemented by special purpose hardware-based computer systems which perform the specified functions, or combinations of special purpose hardware and computer instructions. The sequence diagram 700 of FIG. 7 is used for the intraoral scan registration. Fewer, more, or different steps may be provided.

The handheld intraoral scanning device 104 may be further configured to monitor a bandwidth and connection of the communication channels 108. Based on the monitoring, the handheld intraoral scanning device 104 may perform some steps on the generated 3D surface information before transmission of the 3D surface information to the one or more client devices 106 as described in FIG. 8.

FIG. 8 illustrates an example flowchart 800 comprising different cases for transmission of the 3D surface information to the one or more client devices 106, in accordance with an example embodiment. FIG. 8 is explained in conjunction with elements of FIG. 1, FIG. 2, FIG. 3, FIG. 4, FIG. 5, FIG. 6 and FIG. 7. The flowchart 800 may depict operations performed by at least one of the handheld intraoral scanning device 104 and the one or more client devices 106.

At step 802, the plurality of 2D scan images 110 may be captured. The handheld intraoral scanning device 104 may be configured to capture the plurality of 2D scan images 110 of the dental arch 506 of the patient 504 during the scanning session. Details of the capture of the plurality of 2D scan images 110 are further provided, for example, in FIG. 5.

At step 804, the 3D surface information may be provided. The intraoral scanning device 104 may be configured to provide the 3D surface information based on the captured plurality of 2D scan images 110. For example, the 3D surface information may be the 3D point cloud data. Details of providing the 3D surface information are further provided, for example, in FIG. 5.

At step 806, the bandwidth of the one of the one or more wireless full-duplex communication channels may be monitored. In some embodiments, the bandwidth of the one of the one or more wireless full-duplex communication channel may be monitored by the monitoring unit 208 of the handheld intraoral scanning device 104. For example, the bandwidth of the one of the one or more wireless full-duplex communication channel may be monitored to determine a throughput and latency of the one of the one or more wireless full-duplex communication channel.

At step 808, the handheld intraoral scanning device 104 may check if the bandwidth of the one of the one or more wireless full-duplex communication channels is below a minimum bandwidth. In some embodiments, the monitoring unit 208 of the handheld intraoral scanning device 104 may be configured to check if the bandwidth of the one of the one or more wireless full-duplex communication channels is below the minimum bandwidth.

At step 810, based on the determination that the bandwidth of the one of the one or more wireless full-duplex communication channels is above or equal to the minimum bandwidth, the handheld intraoral scanning device 104 may transmit the 3D surface information 510 to the one or more client devices 106. For example, the handheld intraoral scanning device 104 may transmit the 3D surface information 510 to the one or more client devices 106 via the one of the one or more wireless full-duplex communication channels. Details of the transmission of the 3D surface information 510 are further provided, for example, in FIG. 6.

At step 812, based on the determination that the bandwidth of the one of the one or more wireless full-duplex communication channels is below the minimum bandwidth, the 3D surface information 510 may be down-sampled. In some embodiments, the handheld intraoral scanning device 104 may be configured to down-sample the 3D surface information 510 to be transmitted via the one of the one or more wireless full-duplex communication channels. After down-sampling, the 3D surface information 510 may be transmitted to the one or more client devices 106.

In an embodiment, the broadcasted 3D surface information 510 may be down sampled or heavily compressed with a lossy compression to ensure that the one or more client devices 106 receive the 3D surface information 510 in real-time. Moreover, remaining data of the 3D surface information 510 may further be buffered on the handheld intraoral scanning device 104 to be sent later, either during the scanning session or after the scanning session has finished, when the bandwidth of the one or more wireless full-duplex communication channels is above or equal to the minimum bandwidth. In certain cases, the 3D surface information 510 that may require to be sent for further post-processing on a remote server may be buffered by the connected one or more client devices 106. In some embodiments, the plurality of 2D scan images 110 and the 3D surface information 510 may be stored at the handheld intraoral scanning device 104 or the one or more client devices 106 and later sent for the rendering or for post-processing at a remote server either in the dental clinic or the cloud.

In an embodiment, the WebAssembly module may be used for performing some processing on the one or more client devices 106, when the one or more client devices 106 is a computer having the high computational power. In such a manner, a battery of the handheld intraoral scanning device 104 may be saved. Thus, the web application for the scanning may be loaded directly from the handheld intraoral scanning device 104 to any of the one or more client devices 106 without installation.

It may be noted that the 3D surface information 510 needed to render the interactive 3D graphical representation 112 on the one or more client devices 106 during scanning session is intended for visual feedback for the dentist 502 and thus, may be highly compressed even with lossy compression as long as the interactive 3D graphical representation 112 appears complete. Such compression may reduce an amount of data (such as the 3D surface information 510) that needs to be sent over the communication channels 108 during the scanning session. The amount of data to be transmitted may even be adjusted depending on the available bandwidth of the communication channels 108. In case the bandwidth drops below the minimum bandwidth, a less dense data set of the 3D surface information 510 may be sent and when the bandwidth goes above the minimum bandwidth, the remaining data may be transmitted.

At step 814, the 3D surface information 510 may be stored in the temporary storage unit 210. In some embodiments, the temporary storage unit 210 may be configured to store the 3D surface information 510, when the bandwidth of the one of the one or more wireless full-duplex communication channels is determined to be below the minimum bandwidth. The storage of the 3D surface information 510 in the temporary storage unit 210 may enable a fast retrieval of the 3D surface information 510 when required. The monitoring unit 208 may be configured to continuously monitor the bandwidth of the one of the one or more wireless full-duplex communication channels. Based on the determination that the bandwidth of the one of the one or more wireless full-duplex communication channels is above or equal the minimum bandwidth, the temporary storage unit 210 may transmit the 3D surface information 510 to the one or more client devices 106.

At step 816, the handheld intraoral scanning device 104 may be configured to check if the bandwidth of the one of the one or more wireless full-duplex communication channels is below the minimum bandwidth longer than a maximum period. In some embodiments, the monitoring unit 208 may be configured to check if the bandwidth of the one of the one or more wireless full-duplex communication channels is below the minimum bandwidth longer than a maximum period.

Based on the determination that the bandwidth of the one of the one or more wireless full-duplex communication channels is not below the minimum bandwidth longer than the maximum period, the handheld intraoral scanning device 104 may be configured to transmit the 3D surface information 510 to the one or more client devices 106 via the one of the one or more wireless full-duplex communication channels as described at step 810.

At step 818, based on the determination that the bandwidth of the one of the one or more wireless full-duplex communication channels is below the minimum bandwidth longer than the maximum period, the handheld intraoral scanning device 104 may be configured to compress and store the 3D surface information 510 into the memory unit 204 of the handheld intraoral scanning device 104. The 3D surface information 510 may be stored on the memory unit 204 or on a disk associated with the handheld intraoral scanning device 104 that may later be downloaded by the one or more client devices 106 or uploaded to a powerful scan server for post-processing and diagnostics. Further, the 3D surface information 510 may be compressed and stored for later use. Furthermore, as majority of the processing of the 3D surface information 510 may have been done on the handheld intraoral scanning device 104, the intraoral scanning system 102 may be able to remove noise and irrelevant data from the 3D surface information 510 and hence may aid in reduction of a size of the 3D surface information 510 even further.

At step 820, the connection to the one of the communication channels 108 may be monitored. In some embodiments, the monitoring unit 208 may be configured to monitor the connection to the one of the one or more wireless full-duplex communication channels.

At step 822, the monitoring unit 208 may be configured to check whether the connection to the one of the one or more wireless full-duplex communication channels is lost.

Based on the determination that the connection to the one of the one or more wireless full-duplex communication channels not lost, the handheld intraoral scanning device 104 may be configured to transmit the 3D surface information 510 to the one or more client devices 106 via the one of the one or more wireless full-duplex communication channels as described at step 810.

In some embodiments, the handheld intraoral scanning device 104 may be configured to transmit via a wireless communication interface of the intraoral scanning system 102, the stored 3D surface information 510 in the memory unit 204 when the scanning session is finished. After the dental arch 506 has been scanned by the dentist 502, the handheld intraoral scanning device 104 may transmit the 3D surface information 510 to the memory unit 204 via the wireless communication interface of the communication channels 108. For example, the wireless communication interface may be a web network or the full duplex communication channels.

Further, based on the determination that the connection to the one of the one or more wireless full-duplex communication channels is lost, the handheld intraoral scanning device 104 may be configured compress and store the 3D surface information 510 into the memory unit 204 of the handheld intraoral scanning device 104 as described at step 818.

Such monitoring of the connection and storing the 3D surface information 510 may secure the 3D surface information 510 in case the connection is lost. Moreover, in case the one or more client devices 106 runs out of battery, the intraoral scanning system 102 enables the scanning session to continue, thereby providing flexibility in terms of where and when to send the 3D surface information 510. Typically, the real time feedback required by the dentist 502 while scanning the dental arch 506 without any latency may be more important than a resolution of the 3D surface information 510. On the other hand, when the 3D surface information 510 or the 3D model is inspected by the dentist 502 to perform the diagnostics work, the time taken to optimize and the 3D model becomes less critical. Thus, the 3D surface information 510 may easily be sent to the server for postprocessing and the processed 3D surface information 510 may then be sent back to the one or more client devices 106 for rendering either in the dental clinic or on the cloud server.

At step 824, based on the determination that at least one of the bandwidth of the one of the one or more wireless full-duplex communication channels is below the minimum bandwidth or the connection is lost, the monitoring unit 208 may be configured to transmit a status input to the scanning feedback unit 212. In some embodiments, the scanning feedback unit 212 may receive the status input from the monitoring unit 208. The status input may be for example, a control signal transmitted to the scanning feedback unit 212.

At step 826, while receiving the status input from the monitoring unit 208, the scanning feedback unit 212 may be configured to provide a scanning feedback signal to a user (such as the dentist 506) of the handheld intraoral scanning device 104. The scanning feedback signal may be configured to provide guidance to the user to an area of the dental arch 506 where a scanning quality of the scanning session is low and unable to provide the 3D surface information 510. The scanning feedback signal may assist the dentist 504 to capture more number of the plurality of 2D scan images 110.

In some embodiments, the scanning feedback signal may include an acoustic feedback signal configured to guide the user towards the area of the dental arch 506. In an embodiment, the acoustic feedback signal may be output by the speaker 214A of the handheld intraoral scanning device 104. For example, a beep sound may be output by the speaker 214A as the acoustic feedback signal. In another embodiment, a continuous beep sound may be output by the speaker 214A as the acoustic feedback signal. In an exemplary scenario, an intensity of the acoustic feedback signal increases as the handheld intraoral scanning device 104 is moved towards the area where the scanning quality of the scanning session is low. Moreover, the intensity of the acoustic feedback signal decrease as the handheld intraoral scanning device 104 is moved away from the area where the scanning quality of the scanning session is low.

In some embodiments, the scanning feedback signal may include at least one of haptic feedback, or light emitted by the plurality of LEDs 241C of the handheld intraoral scanning device 104. The haptic feedback and/or the emitted light may be utilized by the dentist 502 to guide the handheld intraoral scanning device 104 in the area where the scanning quality of the scanning session is low.

In some embodiments, the handheld intraoral scanning device 104 may include the vibrator 214B that may be configured to provide the haptic feedback. In some cases, an increase in the vibration of the vibrator 214B may indicate an increasing distance between the area of the dental arch 506 where the scanning quality of the scanning session is low and the handheld intraoral scanning device 104. Moreover, a decrease in the vibration of the vibrator 214B may indicate a decreasing distance between the area of the dental arch 506 where the scanning quality of the scanning session is low and the handheld intraoral scanning device 104. Such feedback provides the guidance to the dentist 502 to accurately place the handheld intraoral scanning device 104 in the areas where the scanning quality of the scanning session is low.

In some embodiments, the plurality of LEDs 241C of the handheld intraoral scanning device 104 may be divided into a left group of LEDs and a right group of LEDs. For example, the left group of LEDs may be arranged on a left side of the handheld intraoral scanning device 104, and the right group of LEDs may be arranged on a right side of the handheld intraoral scanning device 104. The left group of LEDs and the right group of LEDs may be configured to emit a flash of light when the handheld intraoral scanning device 104 is arranged at right or left to the area of the dental arch 506, respectively. The flash of light may be utilized by the dentist 502 to accurately place the handheld intraoral scanning device 104 in the areas where the scanning quality of the scanning session is low. Based on the guidance received by the dentist 502, the dentist 502 may further capture a greater number of the plurality of 2D scan images 110.

It will be understood that each step of the flowchart 800 may be implemented by various means, such as hardware, firmware, processor, circuitry, and/or other communication devices associated with execution of software including one or more computer program instructions. For example, one or more of the steps described above may be embodied by computer program instructions. In this regard, the computer program instructions which embody the steps described above may be stored by the memory 204 of the handheld intraoral scanning device 104, employing an embodiment of the present disclosure. As will be appreciated, any such computer program instructions may be loaded onto a computer or other programmable apparatus (for example, hardware) to produce a machine, such that the resulting computer or other programmable apparatus implements the functions specified in the flowchart 800. These computer program instructions may also be stored in a computer-readable memory that may direct a computer or other programmable apparatus to function in a particular manner, such that the instructions stored in the computer-readable memory produce an article of manufacture the execution of which implements the function specified in the flowchart 800. The computer program instructions may also be loaded onto a computer or other programmable apparatus to cause a series of operations to be performed on the computer or other programmable apparatus to produce a computer-implemented process such that the instructions which execute on the computer or other programmable apparatus provide operations for implementing the functions specified in the flowchart 800.

Accordingly, the steps of the flowchart 800 support combinations of means for performing the specified functions and combinations of operations for performing the specified functions for performing the specified functions. It will also be understood that one or more steps of the flowchart 800, and combinations of steps in the flowchart 800, may be implemented by special purpose hardware-based computer systems which perform the specified functions, or combinations of special purpose hardware and computer instructions. The flowchart 800 of FIG. 8 is used for the intraoral scan registration. Fewer, more, or different steps may be provided.

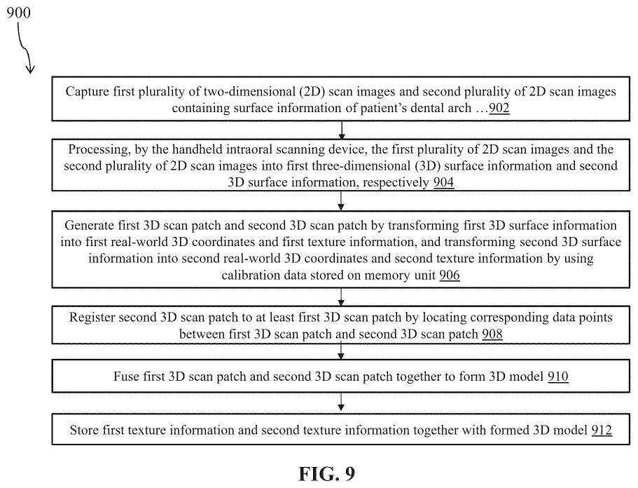

In some embodiments, the handheld intraoral scanning device 104 may be further configured to generate a 3D model representation of the dental arch 506 by combining a plurality of the 3D surface information 510 provided by the handheld intraoral scanning device 104 during the scanning session of the dental arch 506. The handheld intraoral scanning device 104 may further transmit the 3D model via the communication channels 108 to the one or more client devices 106. The generation of the 3D model is further explained in FIG. 9.

FIG. 9 illustrates an example flowchart 900 for generation of the 3D model based on the 3D surface information 510, in accordance with another example embodiment. FIG. 9 is explained in conjunction with elements of FIG. 1, FIG. 2, FIG. 3, FIG. 4, FIG. 5, FIG. 6, FIG. 7 and FIG. 8. The flowchart 900 may depict operations performed by at least one of the handheld intraoral scanning device 104 and the one or more client devices 106.

At step 902, the handheld intraoral scanning device 104 may be configured to capture a first plurality of 2D scan images and a second plurality of 2D scan images containing surface information of the dental arch 506 of the patient 504 during a first time frame and a second time frame, respectively. The first time frame is before the second time frame. For example, the handheld intraoral scanning device 104 captures multiple scan images per sub-scan (such as each plurality of the 2D scan images). The multiple scan images per sub-scan may be combined into raw scan data. The in-built camera of the handheld intraoral scanning device 104 may be moved around the teeth and gums of the patient 504 to capture the first plurality of 2D scan images and the second plurality of 2D scan images.

At step 904, the handheld intraoral scanning device 104 may be configured to process the first plurality of 2D scan images and the second plurality of 2D scan images into first 3D surface information and second 3D surface information, respectively. The first 3D surface information and second 3D surface information may be generated by combining the raw scan data of the first plurality of 2D scan images and the second plurality of 2D scan images. In an embodiment, the processing of the first plurality of 2D scan images and the second plurality of 2D scan images may be based on focus scanning.

The scanning device preferably further comprises optical components for directing the light from the light source to the surface of the dental object. The specific arrangement of the optical components depends on whether the scanning device is a focus scanning apparatus, a scanning device using triangulation principle, confocal scanning, depth of defocus, light field, stereoscopic, deep learning based 3D measurements or any other type of scanning device. The light reflected from the dental object in response to the illumination of the dental object is directed, using optical components of the scanning device, towards the image sensor(s). The image sensor(s) are configured to generate a plurality of images based on the incoming light received from the illuminated dental object. The image sensor may be a high-speed image sensor such as an image sensor configured for acquiring images with exposures of less than 1/1000 second or frame rates in excess of 250 frames pr. second (fps). As an example, the image sensor may be a rolling shutter (CCD) or global shutter sensor (CMOS). For example, an in-focus measurement of the first plurality of 2D scan images and the second plurality of 2D scan images may be performed to generate the first 3D surface information and second 3D surface information respectively. In another embodiment, the handheld intraoral scanning device 104 may utilize a triangulation method to generate the first 3D surface information and second 3D surface information. For example, the handheld intraoral scanning device 104 may project a time varying illumination pattern, where different patterns are projected on the object to be scanned while individual 2D images of the different reflected pattern configurations are recorded. The time varying structured light pattern may be in the form of a gray coded sequence and a phase shifting sequence of stripes, bars or checkers. then the projected features may be tracked across the first plurality of 2D scan images and the second plurality of 2D scan images, and solve a correspondence between the projected features to triangulate depth information to generate the first 3D surface information and second 3D surface information.

At 906, the handheld intraoral scanning device 104 may be configured to generate a first 3D scan patch by transforming the first 3D surface information into first real-world 3D coordinates and first texture information by using the calibration data stored on the memory unit 204. The handheld intraoral scanning device 104 may further generate a second 3D scan patch by transforming the second 3D surface information into second real-world 3D coordinates and second texture information by using the calibration data stored on the memory unit 204. For example, the calibration data of the in-built camera may be utilized to transform the first 3D surface information and the second 3D surface information. The first real-world 3D coordinates and the first texture information may be extracted from the first plurality of 2D scan images to generate the first 3D scan patch. This may be done by adding a Bayer filter to the image sensor of the scanning device, such that RGB values may be extracted directly from the 2D images if the probe light of the scanner is a multi-chromatic white light source (e.g. board wavelength spectrum from 400-750 nm). Alternatively, color texture may be derived from a set of 2D images by using a monochromatic image sensor (no color filter) by switching rapidly between one or more light monochromatic light sources (e.g. within less then 200 ms), such as a red, green and blue light source. Then the intentities recorded by the sensor can be combined and converted into a RGB image. Similarly, the second real-world 3D coordinates and the second texture information may be extracted from the second plurality of 2D scan images to generate the second 3D scan patch.

At step 908, the handheld intraoral scanning device 104 may be configured to register the second 3D scan patch to at least the first 3D scan patch by locating corresponding data points between the first 3D scan patch and the second 3D scan patch. For example, the data points between the first 3D scan patch and the second 3D scan patch may be mapped to register the second 3D scan patch to at least the first 3D scan patch.

At step 910, the handheld intraoral scanning device 104 may be configured to fuse the first 3D scan patch and the second 3D scan patch together to form the 3D model. Based on the registration of the second 3D scan patch to at least the first 3D scan patch, the handheld intraoral scanning device 104 may fuse the first 3D scan patch and the second 3D scan patch together to form the 3D model. For example, a spatial transformation between each sub-scan of the 3D point cloud data (such as the first 3D surface information and the second 3D surface information) may be performed and individual 3D point clouds may be stitched together to reconstruct the 3D model. A registration algorithm may be used to first align and/or register the incoming scan data/scan patches to the current 3D representation, then fusion of the new scan patch with the 3D representation may be performed before the next scan patch is generated by the scanner. Registration/registering new scan data/scan patch should be understood as determining the location of said scan patch in the (current) digital 3D representation, whereas fusion/fusing the scan data/scan patch should be understood as making said scan data/scan patch a part of the digital 3D representation. For the registration of scan patches a variant of the Iterated Closest Point (ICP) algorithm may be used.

At step 912, the handheld intraoral scanning device 104 may be configured to store the first texture information and the second texture information together with the formed 3D model. For example, the handheld intraoral scanning device 104 may store the first texture information and the second texture information together with the formed 3D model in the memory unit 204 of the handheld intraoral scanning device 104.

It will be understood that each step of the flowchart 900 may be implemented by various means, such as hardware, firmware, processor, circuitry, and/or other communication devices associated with execution of software including one or more computer program instructions. For example, one or more of the steps described above may be embodied by computer program instructions. In this regard, the computer program instructions which embody the steps described above may be stored by the memory 204 of the handheld intraoral scanning device 104, employing an embodiment of the present disclosure. As will be appreciated, any such computer program instructions may be loaded onto a computer or other programmable apparatus (for example, hardware) to produce a machine, such that the resulting computer or other programmable apparatus implements the functions specified in the flowchart 900. These computer program instructions may also be stored in a computer-readable memory that may direct a computer or other programmable apparatus to function in a particular manner, such that the instructions stored in the computer-readable memory produce an article of manufacture the execution of which implements the function specified in the flowchart 900. The computer program instructions may also be loaded onto a computer or other programmable apparatus to cause a series of operations to be performed on the computer or other programmable apparatus to produce a computer-implemented process such that the instructions which execute on the computer or other programmable apparatus provide operations for implementing the functions specified in the flowchart 900.

Accordingly, the steps of the flowchart 900 support combinations of means for performing the specified functions and combinations of operations for performing the specified functions for performing the specified functions. It will also be understood that one or more steps of the flowchart 900, and combinations of steps in the flowchart 900, may be implemented by special purpose hardware-based computer systems which perform the specified functions, or combinations of special purpose hardware and computer instructions. The flowchart 900 of FIG. 9 is used for the generation of the 3D model. Fewer, more, or different steps may be provided.

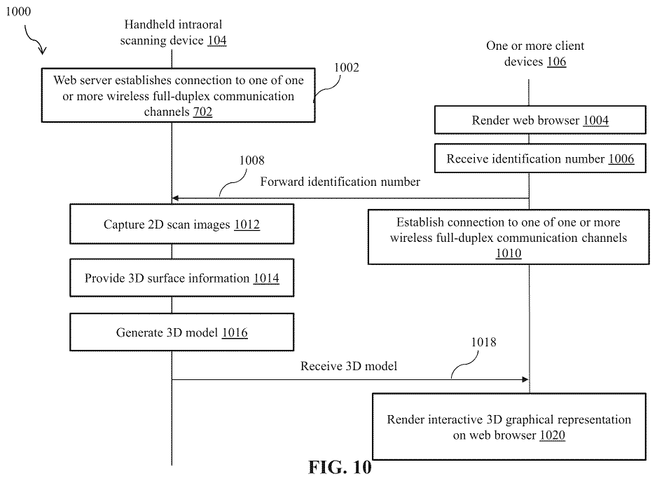

In some embodiments, the handheld intraoral scanning device 104 may be configured to transmit the 3D model to the one or more client devices 106 as described in FIG. 10.

FIG. 10 is a sequence diagram 1000 that depicts render of the interactive 3D graphical representation 112 based on the transmitted 3D model, in accordance with an example embodiment. FIG. 10 is explained in conjunction with elements of FIG. 1, FIG. 2, FIG. 3, FIG. 4, FIG. 5, FIG. 6, FIG. 7, FIG. 8 and FIG. 9. The sequence diagram 1000 may depict operations performed by at least one of the handheld intraoral scanning device 104 and the one or more client devices 106 for transmission of the 3D model.

At step 1002, the web server interface of the handheld intraoral scanning device 104 may establish the connection with one of the communication channels 108. For example, the web server interface of the handheld intraoral scanning device 104 may communicate via the web network to establish the connection with the one or more wireless full-duplex communication channels, as described at step 702 of FIG. 7.

At step 1004, the web browser 602 may be rendered on the one or more client devices 106. For example, the web browser 602 may be rendered on the client device 404, such as the tablet, as described at step 704 of FIG. 7.

At step 1006, the identification number may be received. The identification number may be received via the rendered web browser on the one or more client devices 106, such as the client device 404, as described at step 706 of FIG. 7.

At step 1008, the identification number may be forwarded to the handheld intraoral scanning device 104. The one or more client devices 106 may forward the identification number to the handheld intraoral scanning device 104 input by the user, such as the dentist 502 as described at step 708 of FIG. 7.

At step 1010, the connection to the one of the one or more wireless full-duplex communication channels may be established. Based on the forwarded identification number via the web network, the connection of the one or more client devices 106 to the one of the one or more wireless full-duplex communication channels may be established, as described at step 710 of FIG. 7.

At step 1012, the plurality of 2D scan images 110 may be captured. The handheld intraoral scanning device 104 may be configured to capture the plurality of 2D scan images 110 of the dental arch 506, as described at step 712 of FIG. 7.

At step 1014, the 3D surface information 510 may be provided. The handheld intraoral scanning device 104 may be configured to provide the 3D surface information based on the captured plurality of 2D scan images 110, as described at step 714 of FIG. 7.

At step 1016, the 3D model may be generated. The handheld intraoral scanning device 104 may be configured to generate the 3D model based on the 3D surface information 510. Details of the generation of the 3D model are further provided, for example, in FIG. 9.

At step 1018, the one or more client devices 106 may be configured to receive the 3D model from the intraoral scanning device 104. The 3D model may be received by the one or more client devices 106 by use of the one of the communication channels 108.

At step 1020, the interactive 3D graphical representation 112 may be rendered. The one or more client devices 106 may be configured to render the interactive 3D graphical representation 112 based on the 3D model. In an embodiment, the one or more client devices 106 may render the interactive 3D graphical representation 112 compatible to the web browser 602 independently. Details of render of the interactive 3D graphical representation 112 are further provided, for example, in FIG. 6.

It will be understood that each step of the sequence diagram 1000 may be implemented by various means, such as hardware, firmware, processor, circuitry, and/or other communication devices associated with execution of software including one or more computer program instructions. For example, one or more of the steps described above may be embodied by computer program instructions. In this regard, the computer program instructions which embody the steps described above may be stored by the memory 204 of the handheld intraoral scanning device 104, employing an embodiment of the present disclosure. As will be appreciated, any such computer program instructions may be loaded onto a computer or other programmable apparatus (for example, hardware) to produce a machine, such that the resulting computer or other programmable apparatus implements the functions specified in the sequence diagram 1000. These computer program instructions may also be stored in a computer-readable memory that may direct a computer or other programmable apparatus to function in a particular manner, such that the instructions stored in the computer-readable memory produce an article of manufacture the execution of which implements the function specified in the sequence diagram 1000. The computer program instructions may also be loaded onto a computer or other programmable apparatus to cause a series of operations to be performed on the computer or other programmable apparatus to produce a computer-implemented process such that the instructions which execute on the computer or other programmable apparatus provide operations for implementing the functions specified in the sequence diagram 1000.

Accordingly, the steps of the sequence diagram 1000 support combinations of means for performing the specified functions and combinations of operations for performing the specified functions for performing the specified functions. It will also be understood that one or more steps of the sequence diagram 1000, and combinations of steps in the sequence diagram 1000, may be implemented by special purpose hardware-based computer systems which perform the specified functions, or combinations of special purpose hardware and computer instructions. The sequence diagram 1000 of FIG. 10 is used for the generation and transmission of the 3D model. Fewer, more, or different steps may be provided.

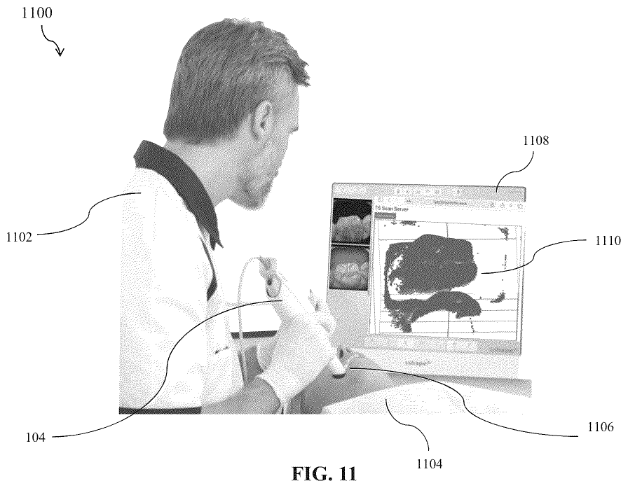

FIG. 11 is a schematic diagram 1100 that depicts an exemplary environment for capture of the plurality of 2D scan images 110 and render of an interactive 3D graphical representation 1110 in real-time, in accordance with an example embodiment. FIG. 11 is explained in conjunction with elements of FIG. 1, FIG. 2, FIG. 3, FIG. 4, FIG. 5, FIG. 6, FIG. 7, FIG. 8, FIG. 9 and FIG. 10. The schematic diagram 1100 may include a dentist 1102 and a patient 1104.

The handheld intraoral scanning device 104 may be utilized by the dentist 1102 to capture the plurality of 2D scan images 110 of a dental arch 1106 of the patient 1104. The handheld intraoral scanning device 104 may process the captured plurality of 2D scan images 110 and generate the 3D surface information 510 of the dental arch 1106 of the patient 1104.

The dentist 1102 may input the identification number for the handheld intraoral scanning device 104 on the web browser of a computer 1108 (such as a client device). The handheld intraoral scanning device 104 and the computer 1108 may be connected on the common web network and via the one of the communication channels 108, based on the identification number forwarded to the handheld intraoral scanning device 104.

Once the handheld intraoral scanning device 104 and the computer 1108 are connected, the 3D surface information 510 of the dental arch 1106 may be transmitted to the computer 1108. The web application may be rendered on the web browser of the computer 1108. The plurality of 2D scan images 110 captured by the dentist 1102 may be displayed on the web application. Further, the interactive 3D graphical representation 1110 may be generated in the real-time based on the 3D surface information 510 and rendered on the web application. The interactive 3D graphical representation 1110 may be manipulated, such as rotated or viewed in multiple perspectives by the dentist 1102 as required.

Thus, the intraoral scanning system 102 may enable the processing of the plurality of 2D scan images 110 independent of any external devices during scan registration. The users, such as the dentists may be able to get the visual feedback during scanning session, and access the web server interface on the handheld intraoral scanning device 104 by the one or more client devices 106. Further, the 3D surface information 510 may be broadcast to render the interactive 3D graphical representation 1110 on the one or more client devices 106. As the 3D surface information 510 may be stored on the handheld intraoral scanning device 104, the user may be able to switch to different client devices while scanning. If connection is lost between the handheld intraoral scanning device 104 and the one or more client devices 106, the scanning session may be resumed without any loss in the scan data.

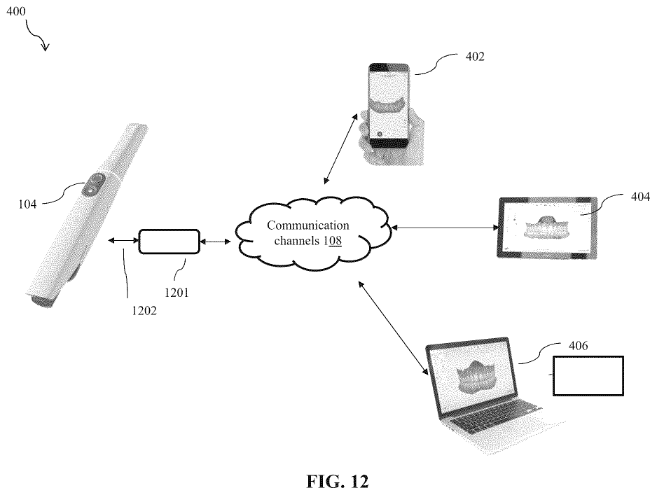

FIG. 12 is a schematic diagram 400 that illustrates an environment for communication of the handheld intraoral scanning device 104 and the one or more client devices 106, in accordance with an example embodiment. FIG. 12 is explained in conjunction with elements of FIG. 1, FIG. 2, FIG. 3, and FIG. 4. In this present example, the schematic diagram 400 may an intermediate processing unit 1201 configured to communicate with the handheld intraoral scanning 104 via a wireless link 1202 and the one or more client devices (402, 404, 406) via the communication channels 108. The wireless link 1202 may be based on a Bluetooth protocol or WIFI protocol. The wireless link 1202 has a larger bandwidth than the communication channels 108. The intermediate processing unit is configured to process partly the 3D surface information received from the handheld intraoral scanning device for the purpose of reducing the needed bandwidth of the communication channels 108. The partly processing of the 3D surface information may involve compression of the information according to a compression protocol, such as H.265 which is a high efficiency video coding operation. The partly processing of the 3D surface information may further include deletion of 3D surface information that is irrelevant for a 3D model representation of the dental arch.

Since the scan registration and rendering may no longer depend on a high computational computer, such as the power PC, the power PC or the server may solely be used for heavy computations. Moreover, the intraoral scanning system 102 enables handling of processing of multiple scans in parallel. The number of scans that may be post-processed simultaneously may thus only depend on the processing power of the single server and there is thus not necessarily any need for multiple high computational computers. Further, the post-processing may be delegated to a server in the cloud. Thus, the intraoral scanning system 102 provides the intraoral scan registration by use of the edge computing technique, by bringing the processing in the handheld intraoral scanning device 104.

Many modifications and other embodiments of the inventions set forth herein will come to mind of one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

### Item List

1. An intraoral scanning system (102) comprising:
   a handheld intraoral scanning device (104) configured to:
      capture a plurality of two-dimensional (2D) scan images (110) during a scanning session of a dental arch (506);
      provide three-dimensional (3D) surface information (510) based on the plurality of 2D scan images (110) captured during the scanning session, and
         wherein the handheld intraoral scanning device (104) comprises a web server interface (206) configured to communicate via a web network and establish a connection to one or more wireless full-duplex communication channels; and
   one or more client devices (106), wherein each of the one or more client devices (106) is configured to:
      establish a connection to one of the one or more wireless full-duplex communication channels by forwarding an identification number to the handheld intraoral scanning device (104) via the web network;
      receive the 3D surface information (510) via the one of the one or more wireless full-duplex communication channels; and
      render the 3D surface information (510) into an interactive 3D graphical representation (112) compatible to a web browser (602).
2. The intraoral scanning system (102) according to item 1, wherein the web server interface (206) and the one or more client devices (106) are connected to a common web network.
3. The intraoral scanning system (102) according to any of the previous items, wherein multiple of the one or more client devices (106) are configured to:
   receive the 3D surface information (510) via a wireless full-duplex communication channel of the one or more wireless full-duplex communication channels; and
   render the 3D surface information (510) into the interactive 3D graphical representation (112) compatible to the web browser (602).
4. The intraoral scanning system (102) according to any of the previous items, wherein the one or more client devices (106) is at least one of: a displaying unit, a tablet, or a smartphone.
5. The intraoral scanning system (102) according to any of items 1 to 3, wherein the one or more client devices (106) is a computer.
6. The intraoral scanning system (102) according to any of the previous items, wherein a bandwidth of the one of the one or more wireless full-duplex communication channels is monitored by a monitoring unit (208) of the intraoral scanning system (102), and
   when the bandwidth is below a minimum bandwidth, the handheld intraoral scanning device (104) is configured to down-sample the 3D surface information (510) to be transmitted via the one of the one or more wireless full-duplex communication channels.
7. The intraoral scanning system (102) according to item 6, wherein the bandwidth of the one of the one or more wireless full-duplex communication channels is monitored by the monitoring unit (208) of the intraoral scanning system (102), and
the handheld intraoral scanning device (104) comprises a temporary storage unit (210) configured to:
   store the 3D surface information (510), when the bandwidth of the one of the one or more wireless full-duplex communication channels is determined to be below the minimum bandwidth; and
   transmit the stored 3D surface information (510), when the bandwidth is determined to be above or equal the minimum bandwidth.
8. The intraoral scanning system (102) according to item 7, wherein when the bandwidth of the one of the one or more wireless full-duplex communications is below the minimum bandwidth longer than a maximum period, the handheld intraoral scanning device (104) is configured to compress and store the 3D surface information (510) into a memory unit (204) of the handheld intraoral scanning device (104).
9. The intraoral scanning system (102) according to item 7, wherein
   the monitoring unit (208) is configured to determine when a connection to the one of the one or more wireless full-duplex communication channels is lost; and
   the handheld intraoral scanning device (104) is configured to compress and store the 3D surface information (510) into a memory unit (204) of the handheld intraoral scanning device (104), based on the determination that the connection is lost.
10. The intraoral scanning system (102) according to item 9, wherein the handheld intraoral scanning device (104) is configured to transmit via a wireless communication interface of the intraoral scanning system (102), the stored 3D surface information (510) in the memory unit (204) when the scanning session is finished.
11. The intraoral scanning system (102) according to any of items 7 to 10, wherein the handheld intraoral scanning device (104) further comprises:
   the monitoring unit (208) that is configured to transmit a status input based on at least one of: the determination that the bandwidth is less than the minimum bandwidth, or the determination that the connection to the one of the one or more wireless full-duplex communication channels is lost, and
   a scanning feedback unit (212) configured to:
      receive the status input from the monitoring unit (208), and
      provide a scanning feedback signal to a user of the handheld intraoral scanning device (104), while receiving the status input, wherein the scanning feedback signal is configured to provide guidance to the user to an area of the dental arch (506) where a scanning quality of the scanning session is low and unable to provide the 3D surface information (510).
12. The intraoral scanning system (102) according to item 11, wherein the scanning feedback signal includes an acoustic feedback signal configured to guide the user towards the area of the dental arch (506).
13. The intraoral scanning system (102) according to item 11, wherein the scanning feedback signal comprises at least one of: haptic feedback, or light emitted by a plurality of light emitting diodes of the handheld intraoral scanning device (104).
14. The intraoral scanning system (102) according to item 13, wherein the handheld intraoral scanning device (104) comprises a vibrator (214B) configured to provide the haptic feedback, and wherein an increase in the vibration indicates an increasing distance between the area of the dental arch (506) and the handheld intraoral scanning device (104), and wherein a decrease in the vibration indicates a decreasing distance between the area of the dental arch (506) and the handheld intraoral scanning device (104).
15. The intraoral scanning system (102) according to item 13, wherein the plurality of light emitting diodes (214° C.) is divided into a left group of light emitting diodes and a right group of light emitting diodes, and
   wherein the left group and the right group are configured to emit a flash of light when the handheld intraoral scanning device (104) is arranged at right or left to the area of the dental arch (506), respectively.
16. The intraoral scanning system (102) according to any of the previous items, wherein the handheld intraoral scanning device (104) is configured to broadcast the 3D surface information (510) to multiple of the one or more client devices (106) connected to the handheld intraoral scanning device (104) via the one of the one or more wireless full-duplex communication channels.
17. The intraoral scanning system (102) according to any of the previous items, wherein the handheld intraoral scanning device (104) is configured to generate a 3D model representation of the dental arch (506) by combining a plurality of the 3D surface information (510) provided by the handheld intraoral scanning device (104), and wherein
   the handheld intraoral scanning device (104) is configured to transmit the 3D model via the one of the one or more wireless full-duplex communication channels.
18. A method (700) for intraoral scan registration comprising:
   capturing (712), by a handheld intraoral scanning device (104), a plurality of two-dimensional (2D) scan images (110) during a scanning session of a dental arch (506);
   providing (714), by the handheld intraoral scanning device (104), three-dimensional (3D) surface information (510) based on the plurality of 2D scan images (110) captured during the scanning session;
   establishing (710), by one or more client devices (106), a connection to one of one or more wireless full-duplex communication channels by forwarding (708) an identification number to the handheld intraoral scanning device (104) via a web network;
   receiving (716), by the one or more client devices (106), the 3D surface information (510) via the one of the one or more wireless full-duplex communication channels; and
   rendering (718), on the one or more client devices (106), the 3D surface information (510) into an interactive 3D graphical representation (112) compatible to a web browser (602).
19. An intraoral scanning system (102) comprising:
   a handheld intraoral scanning device (104) configured to:
      capture a first plurality of two-dimensional (2D) scan images and a second plurality of 2D scan images containing surface information of a patient's dental arch (506) during a first time frame and a second time frame, respectively, and wherein the first time frame is before the second time frame;
      process the first plurality of 2D scan images and the second plurality of 2D scan images into first three-dimensional (3D) surface information and second 3D surface information, respectively;
      generate a first 3D scan patch and a second 3D scan patch by transforming the first 3D surface information into first real-world 3D coordinates and first texture information, and transforming the second 3D surface information into second real-world 3D coordinates and second texture information by using calibration data stored on a memory unit (204);
      register the second 3D scan patch to at least the first 3D scan patch by locating corresponding data points between the first 3D scan patch and the second 3D scan patch;
      fuse the first 3D scan patch and the second 3D scan patch together to form a 3D model;
      store the first texture information and the second texture information together with the formed 3D model, and
         wherein the handheld intraoral scanning device (104) comprises a web server interface (206) configured to communicate via a web network and establish a connection to one or more wireless full-duplex communication channels; and
   one or more client devices (106), wherein each of the one or more client devices (106) is configured to:
      establish a connection to one of the one or more wireless full-duplex communication channels by forwarding an identification number to the handheld intraoral scanning device (104) via the web network;
      receive the 3D model via the one of the one or more wireless full-duplex communication channels; and
      render the 3D model into an interactive 3D graphical representation (112) compatible to a web browser (602).
20. A method for intraoral scan registration comprising:
   capturing, by a handheld intraoral scanning device (104), a first plurality of two-dimensional (2D) scan images and a second plurality of 2D scan images containing surface information of a patient's dental arch (506) during a first time frame and a second time frame, respectively, and wherein the first time frame is before the second time frame;
   processing, by the handheld intraoral scanning device (104), the first plurality of 2D scan images and the second plurality of 2D scan images into first three-dimensional (3D) surface information and second 3D surface information, respectively;
   generating, by the handheld intraoral scanning device (104), a first 3D scan patch and a second 3D scan patch by transforming the first 3D surface information into first real-world 3D coordinates and first texture information, and transforming the second 3D surface information into second real-world 3D coordinates and second texture information by using calibration data;
   registering, by the handheld intraoral scanning device (104), the second 3D scan patch to at least the first 3D scan patch by locating corresponding data points between the first 3D scan patch and the second 3D scan patch;
   fusing, by the handheld intraoral scanning device (104), the first 3D scan patch and the second 3D scan patch together to form a 3D model;
   storing, by the handheld intraoral scanning device (104), the first texture information and the second texture information together with the formed 3D model;
   establishing, by one or more client devices, a connection to one of one or more wireless full-duplex communication channels by forwarding an identification number to the handheld intraoral scanning device via a web network;
   receiving, by the one or more client devices, the 3D model via the one of the one or more wireless full-duplex communication channels; and
   rendering, on the one or more client devices, the 3D model into an interactive 3D graphical representation (112) compatible to a web browser (602).
21. A computer programmable product comprising a non-transitory computer readable medium having stored thereon computer executable instructions, which when executed by a processing circuitry, cause the processing circuitry to carry out operations, the operations comprising:
   capturing (902) a first plurality of two-dimensional (2D) scan images and a second plurality of 2D scan images containing surface information of a patient's dental arch (506) during a first time frame and a second time frame, respectively, and wherein the first time frame is before the second time frame;
   processing (904) the first plurality of 2D scan images and the second plurality of 2D scan images into first three-dimensional (3D) surface information and second 3D surface information, respectively;
   generating (906) a first 3D scan patch and a second 3D scan patch by transforming the first 3D surface information into first real-world 3D coordinates and first texture information, and transforming the second 3D surface information into second real-world 3D coordinates and second texture information by using calibration data;
   registering (908) the second 3D scan patch to at least the first 3D scan patch by locating corresponding data points between the first 3D scan patch and the second 3D scan patch;
   fusing (910) the first 3D scan patch and the second 3D scan patch together to form a 3D model; and
   storing (912) the first texture information and the second texture information together with the formed 3D model.

1. An intraoral scanning system comprising:
a handheld intraoral scanning device configured to:
capture a plurality of two-dimensional scan images during a scanning session of a dental arch;
provide three-dimensional surface information based on the plurality of 2D scan images captured during the scanning session, and
wherein the handheld intraoral scanning device comprises a web server interface configured to communicate via a web network and establish a connection to one or more wireless full-duplex communication channels; and
one or more client devices, wherein each of the one or more client devices is configured to:
establish a connection to one of the one or more wireless full-duplex communication channels by forwarding an identification number to the handheld intraoral scanning device via the web network;
receive the 3D surface information via the one of the one or more wireless full-duplex communication channels; and
render the 3D surface information into an interactive 3D graphical representation compatible to a web browser.

2. The intraoral scanning system according to claim 1, wherein the web server interface and the one or more client devices are connected to a common web network.

3. The intraoral scanning system according to claim 1, wherein multiple of the one or more client devices are configured to:
receive the 3D surface information via a wireless full-duplex communication channel of the one or more wireless full-duplex communication channels; and
render the 3D surface information into the interactive 3D graphical representation compatible to the web browser.

4. The intraoral scanning system according to claim 1, wherein the one or more client devices is at least one of: a displaying unit, a tablet, or a smartphone.

5. The intraoral scanning system according to claim 1, wherein the one or more client devices is a computer.

6. The intraoral scanning system according to claim 1, a bandwidth of the one of the one or more wireless full-duplex communication channels is monitored by a monitoring unit of the intraoral scanning system and
when the bandwidth is below a minimum bandwidth, the handheld intraoral scanning device is configured to down-sample the 3D surface information to be transmitted via the one of the one or more wireless full-duplex communication channels.

7. The intraoral scanning system according to claim 6, wherein the bandwidth of the one of the one or more wireless full-duplex communication channels is monitored by the monitoring unit of the intraoral scanning system, and the handheld intraoral scanning device comprises a temporary storage unit configured to:
store the 3D surface information , when the bandwidth of the one of the one or more wireless full-duplex communication channels is determined to be below the minimum bandwidth; and
transmit the stored 3D surface information when the bandwidth is determined to be above or equal the minimum bandwidth.

8. The intraoral scanning system according to claim 7, wherein when the bandwidth of the one of the one or more wireless full-duplex communications is below the minimum bandwidth longer than a maximum period, the handheld intraoral scanning device is configured to compress and store the 3D surface information into a memory unit of the handheld intraoral scanning device.

9. The intraoral scanning system according to claim 7, wherein
the monitoring unit is configured to determine when a connection to the one of the one or more wireless full-duplex communication channels is lost; and
the handheld intraoral scanning device is configured to compress and store the 3D surface information into a memory unit of the handheld intraoral scanning device, based on the determination that the connection is lost.

10. The intraoral scanning system according to claim 9, wherein the handheld intraoral scanning device is configured to transmit via a wireless communication interface of the intraoral scanning system, the stored 3D surface information in the memory unit when the scanning session is finished.

11. The intraoral scanning system according to claim 7, wherein the handheld intraoral scanning device further comprises:
the monitoring unit that is configured to transmit a status input based on at least one of: the determination that the bandwidth is less than the minimum bandwidth, or the determination that the connection to the one of the one or more wireless full-duplex communication channels is lost, and
a scanning feedback unit configured to:
receive the status input from the monitoring unit, and
provide a scanning feedback signal to a user of the handheld intraoral scanning device, while receiving the status input, wherein the scanning feedback signal is configured to provide guidance to the user to an area of the dental arch where a scanning quality of the scanning session is low and unable to provide the 3D surface information.

12. The intraoral scanning system according to claim 1, wherein the handheld intraoral scanning device is configured to broadcast the 3D surface information to multiple of the one or more client devices connected to the handheld intraoral scanning device via the one of the one or more wireless full-duplex communication channels.

13. The intraoral scanning system according to claim 1, wherein the handheld intraoral scanning device is configured to generate a 3D model representation of the dental arch by combining a plurality of the 3D surface information provided by the handheld intraoral scanning device, and wherein
the handheld intraoral scanning device is configured to transmit the 3D model via the one of the one or more wireless full-duplex communication channels.

14. A method for intraoral scan registration comprising:
capturing, by a handheld intraoral scanning device, a plurality of two-dimensional scan images during a scanning session of a dental arch;
providing, by the handheld intraoral scanning device, three-dimensional surface information based on the plurality of 2D scan images captured during the scanning session;
establishing, by one or more client devices, a connection to one of one or more wireless full-duplex communication channels by forwarding an identification number to the handheld intraoral scanning device via a web network;
receiving, by the one or more client devices, the 3D surface information via the one of the one or more wireless full-duplex communication channels; and
rendering, on the one or more client devices, the 3D surface information into an interactive 3D graphical representation compatible to a web browser.

15. The method according to claim 14, comprising connecting the web server interface and the one or more client devices to a common web network.
